(19) United States
(12) Patent Application Publication
(10) Pub. No.: US 2018/0160709 A1
(43) Pub. Date: Jun. 14, 2018

(54) bitter taste removing preparation and method

(71) Applicants: Zhongyuan Zhu, The Woodlands, TX (US); Fanfeng Ma, The Woodlands, TX (US); Mu Zhu, Suzhou (CN); Xiyuan Zhu, Suzhou (CN)

(72) Inventors: Zhongyuan Zhu, The Woodlands, TX (US); Fanfeng Ma, The Woodlands, TX (US); Mu Zhu, Suzhou (CN); Xiyuan Zhu, Suzhou (CN)

(21) Appl. No.: 15/836,818

(22) Filed: Dec. 8, 2017

(30) Foreign Application Priority Data
Dec. 8, 2016 (CN) 2016111234416

(51) Int. Cl. A23L 27/00; A23L 27/20; A61K 38/47; A61K 9/00; A61K 9/68

(52) U.S. Cl. CPC A23L 27/86; A23L 27/20; A61K 38/47; A23V 2002/00; A61K 9/0058; A61K 9/006; A61K 9/0095; A61K 9/0056

(57) ABSTRACT

The current invention relates to a gargle dosage formulation and a method for removing the bitter taste in oral medication or food. Particularly, this bitter-removal formulation comprises at least one of two preparations, the first preparation and the second preparation. The current invention also discloses a method of using the preparations before and after taking oral medication or food. Right before taking oral medication or food, the first preparation is used to gargle the user's oral cavity. After it is spat out, the first preparation forms a thin layer on the surface of the oral mucosa to cover, shield, and protect taste buds from the direct contact with oral medication or food. Immediately after oral medication or food is taken, the second preparation is used to gargle and clean the user's taste buds, which effectively elutes mucosa-binding bitter ingredients from the oral cavity. Because both preparations are spat out, there is neither body intake of these preparations nor interference to the oral medication. The gargle preparation can be generally used with various types of oral medication. Both preparations in this formulation are simple, safe, inexpensive, and easy to use. The first and second preparations may be used together or only one of them is used. The gargle preparation is very effective to minimize and remove tastes such as the bitter taste, peculiar taste, and dulled taste, which are associated with the traditional herbal medicine such as traditional Chinese herbs and their decoctions. The gargle preparation helps patients who are taking oral medication with the bitter taste to maintain healthy taste buds and improve life quality.

### BACKGROUND OF THE INVENTION

Water decoction of herbal medicine is one of the most common forms of traditional Chinese medicine. It is easy to be prepared by patients at home before it is orally taken. When there is a need for changing its dose or ingredients, a doctor of Chinese medicine can quickly adjust the amount of herbal medicine in the formula. Herbal decoction of traditional Chinese medicine is very popular and has been widely used for chronic disease treatment and preventive maintenance. A formula of herbal medicine consists of various plants and each of them has many different ingredients and chemicals, which come out from the herbal plants into the water during the preparation. Many of these ingredients have different kinds of unpleasant tastes such as bitter, astringent, or peculiar taste. All of these tastes make water decoction of the formula have a bitter, astringent, or peculiar taste. The overall taste is generally referred as a bitter taste. Historically, there is a saying in China: “A good medicine always comes with a bitter taste”. However, even though people always welcome a good medicine, it is hard for them to say that they also like its bitter taste. In fact, the bitter taste of herbal medicine has been the complaint by patients for thousands of years. It lowers patients' acceptance level to water decoction and hinders the promotion of herbal medicine in public. Even for those patients who can manage to finish the treatment course of herbal medicine, they may still suffer dulled taste buds and loss of appetite due to the bitter taste.

Previous research has found that the decoction of herbal medication at around 37° C. would have the maximum amount of side effect on taste. Therefore, once the decoction is cooled down to a temperature at which it is not too hot to drink, it should be taken right away to avoid the bitter taste as much as possible. However, people generally prefer to have the decoction when it is cooled down near the room temperature.

In order to remove the bitter taste, some studies had found that some drugs are able to weaken the links between the bitter taste bud and the brain. Other scientists had tried to add taste masking or mucilage agent to the medicine. Putting the medicine into a coated tablet or capsule is also a way to separate the bitter taste from the taste buds in oral cavity. However, all these methods come with additional cost and other side effects. The drug used to block the bitter taste may cause unnecessary side effect to the patient. The masking method introduces new ingredients to the formulation, which may not be compatible to the medicine. The coating method not only needs to redesign the formulation but also add cost to the manufacture process. This method can't be used for water decoction of herbal medicine. Therefore, these methods are not practical and unable to help remove the bitter taste effectively.

It is very much needed to develop a simple, effective, and safe preparation and method for bitter taste removal. This ideal preparation and method should be simple to prepare and easy to use. In addition, this preparation should not interfere with the ingredients existing in herbal or oral medicine. It can also be generally applied for removal of bitter tastes caused by different types of oral medicine. As to the outcome, this preparation should quickly and effectively remove bitter tastes, restore the normal mouth feeling, and finally improve life quality for patients.

### SUMMARY OF THE INVENTION

The goal of the current study is to provide a quick-acting and high efficient bitter taste removal (BTR) preparations and appropriate BTR methods for dispelling the bitter tastes caused by oral medicine or food. This goal is accomplished by the present invention disclosed as follows.

The first aspect of the invention is to provide a BTR agent. The preferred BTR agent comprises a gargle preparation, which further comprises two parts, the first preparation and the second preparation.

The first preparation was suitable for use right before orally taking a medicine or food with bitter taste. The first preparation can shield and cover the taste buds with protective thin layer films formed and spread on the mucosal surface in the oval cavity, which reduce or minimize the direct contact between taste buds and the bitter-inducing factors in the medicine or food.

The second preparation was suitable to be used right after orally taking a medicine or food with bitter taste. It facilitates the bitter-inducing factors to dissociate themselves from the taste buds and the oral mucosal surface before being eluted quickly from the oral cavity.

Both the first preparation and the second preparation are agents for gargling and have to be spat out after several minutes of gargling.

The preferred gargle preparations protect the taste buds from the direct and continuous contact with medicines with bitter taste, thereby maintaining the health of taste buds.

In some particularly preferred embodiments, the components of the first preparations and the second preparation could be the same or different.

In some preferred embodiments, wherein the gargle preparation is used for the medicine or food with bitter taste, which can induce abnormal taste feeling or taste side effect in oral cavity. The medicine or food with bitter taste includes but is not limited to oral medicines with defined molecular structures, traditional herbal medicine, food, edible traditional nutrients, or their ingredients.

In some preferred embodiments, wherein the gargle preparation is used for removal of the said bitter taste in the said medicines or food, which is referred to an abnormal taste in oral cavity includes but is not limited to one or more of the following tastes: bitter taste, peculiar taste, odd taste, queer taste, pungent taste, hot and spicy taste, bad breath, garlic/onion taste, jaded palates, and temporary dulled taste, or other side effects on taste.

In some preferred embodiments, both the first preparation and the second preparation in the gargle preparation comprise excipients used in pharmaceuticals or food.

In some preferred embodiments, the first preparation comprises at least one of the ingredients from the following group: pharmaceutical-grade protein, lipophilic agent, mucosal adhesive, polysaccharide gum, oil-water emulsion, cellulose derivative, adsorbent, pharmaceutical hydrogel, oily gel, and patch for oral use. The second formulation comprises at least one of the ingredients from the following group: pharmaceutical-grade acid, lipophilic agent, pharmaceutical-grade protein and enzyme, mucosal adhesive, cellulose derivatives, oil-water emulsion, adsorbent, surfactant, effervescent agent, polyvinyl pyrrolidone, and pharmaceutical gel base material.

In some particularly preferred embodiments, wherein the pharmaceutical-grade protein in the gargle preparation is selected from the group of whey protein, sodium caseinate, chicken egg albumin, bovine serum albumin, milk powder, soybean protein, albumin protein, gelatin protein, collagen, and any combination thereof.

In some more particularly preferred embodiments, wherein the amount of the pharmaceutical-grade protein is in the range 0-100 mg, preferably 50-800 mg, better 50-600 mg, and the best 50-400 mg.

In some more particularly preferred embodiments, wherein the gelatin protein is used in its gel form with the weight percentage 1-15%, preferably 2-10%.

In some more particularly preferred embodiments, wherein the gelatin protein is warmed up to form a flowing liquid in the water bath before application and the preferred amount for gargle is in the range 1-10 ml.

In some particularly preferred embodiments, wherein the lipophilic agent is selected from the group of edible oil, essential oil, ester of citric acid, glycerin, alcohol or polyethylene glycol, and any combination thereof.

In some more particularly preferred embodiments, wherein the amount lipophilic agent has a weight percentage 0-30%, preferably 1-25%.

In some particularly preferred embodiments, wherein the mucosal adhesive is selected from the group of chitosan, dextrin, β-cyclodextrin, and any combination thereof.

In some more particularly preferred embodiments, wherein the amount of the mucosal adhesive is in the range 0-1000 mg, preferably 10-300 mg, best 30-100 mg.

In some particularly preferred embodiments, wherein the polysaccharide gum is able to form viscous gel under aqueous conditions.

In some particularly preferred embodiments, wherein the polysaccharide gum is selected from the group of guar bean gum, xanthan gum, carrageenan, sodium alginate, arabic gum, tragacanth gum, pectin, maltodextrin with a low dextrose equivalent (DE) value, locust and sophora bean gum, gellan gum, curdlan, tamarind seed gum, konjac gum, agar, thermal gelations, cyclodextrin, pharmaceutical hydrogel, oily gel, and any combination thereof.

In some more particularly preferred embodiments, wherein the amount of polysaccharide gum is in the range of 0-1000 mg, preferably 10-800 mg, better 20-600 mg, the best 50-500 mg.

In some more particularly preferred embodiments, wherein the amount of the maltodextrin in the first preparation is in the range 0-1000 mg, preferably 100-800 mg, better 200-600 mg.

In some particularly preferred embodiments, wherein the oil-water emulsion contains 20-60 parts of an edible oil such as an essential oil, 30-80 parts of water, 0-15 parts of carboxymethyl cellulose (CMC), and 0-15 parts of a surfactant such as soya lecithin.

In some particularly preferred embodiments, wherein the edible oil has a weight percentage 20-60%, preferably 30-45% in the oil-water emulsion.

In some particularly preferred embodiments, wherein the amount of the oil-water emulsion in the gargle preparation is 0-30 ml, preferably 1-25 ml.

In some more particularly preferred embodiments, wherein in the described the edible oil is packed separately from other components of oil-water emulsion and used to form the emulsion before use.

In some more particularly preferred embodiments, wherein the surfactant is selected from the group of Tween, soya lecithin, sodium lauryl sulfate, Span, polymer alkoxy castor oil, poloxamer, and any combination thereof.

In some more particularly preferred embodiments, wherein the soya lecithin and other phospholipids functioned as an agent to block the bitter taste receptors besides being merely as a surfactant.

In some more particularly preferred embodiments, wherein the Tween is selected from the group of Tween 20, Tween 40, Tween 60, polysorbate 80, and any combination thereof.

In some more particularly preferred embodiments, wherein the Span is selected from the group of Span 20, Span 60, Span 80, and any combination thereof.

In some particularly preferred embodiments, wherein the cellulose derivative is selected from the group of CMC, hydroxypropyl methyl cellulose, methyl cellulose, ethyl cellulose, hydroxyethyl cellulose, hydroxyethyl methylcellulose, hydroxypropyl cellulose, and any combination thereof.

In some particularly preferred embodiments, wherein the amount of cellulose derivative in the gargle preparation is in the range 0-250 mg, preferably 5-200 mg, better 10-100 mg.

In some particularly preferred embodiments, wherein the adsorbent is selected from the group of activated charcoal, activated bamboo charcoal powder, macro-porous resin, silica gel, chemically modified silica gel, magnesium silicate, calcium silicates, diatomaceous earth, zeolites, alumina, and any combination thereof.

In some particularly preferred embodiments, wherein the amount of the adsorbent in the preparation is in the range 0-50 mg, preferably 0-20 mg, better 0-10 mg.

In some particularly preferred embodiments, wherein the pharmaceutical hydrogel in the first preparation is selected from the group of Carbomer (Carbopol), polyvinyl alcohol, acrylic acid and its derivatives, polyurethane, and any combination thereof.

In some particularly preferred embodiments, wherein the amount of the first preparation in the first preparation is in the range 0-500 mg, preferably 100-400 mg.

In some particularly preferred embodiments, wherein the pharmaceutical acid is selected from the group of malic acid, tartaric acid, citric acid, fumaric acid, lactic acid, acetic acid, gluconic acid, maleic acid, succinic acid, hydrochloric acid, phosphoric acid, tannic acid, salts of the said acids, and any combination thereof.

In some particularly preferred embodiments, wherein the pharmaceutical acid in the second preparation has a weight percentage 2-10%, preferably 2-6% when it is used to form an aqueous solution.

In some more particularly preferred embodiments, wherein the pH value of the aqueous solution containing the pharmaceutical acid in the second preparation is in the range 2-9, preferably 3-7 or 6-9.

In some particularly preferred embodiments, wherein the pharmaceutical enzyme in the second preparation is selected from the group of α-glucosidase, β-glucosidase, dextran enzyme, cellulase, hemi-cellulase, amylase, xylanase enzyme, rhamnosidase, galactosidase, lactose enzyme, and any combination thereof.

In some particularly preferred embodiments, wherein the total amount of pharmaceutical protein and enzyme in the gargle preparation is in the range 0-1000 mg, preferably 50-600 mg, better 100-500 mg.

In some particularly preferred embodiments, wherein the amount of pharmaceutical enzyme is in the range 0-400 mg, preferably 50-300 mg, better 100-250 mg.

In some particularly preferred embodiments, wherein the adsorbent in the second preparation is selected from same group of chemicals as the first preparation. However, the amount of the adsorbent in the second preparation is in the range 0-1000 mg, preferably 50-600 mg, better 100-500 mg.

In some particularly preferred embodiments, wherein the oil phase of the oil-water emulsion in the second preparation is the same as the one in the first preparation.

In some particularly preferred embodiments, wherein the polysaccharide gum in the second preparation is selected from the same group as the first preparation. However, the amount of the polysaccharide gum in the second preparation is in the range 0-200 mg, preferably 0-80 mg, better 0-50 mg.

In some particularly preferred embodiments, wherein the mucosal adhesive in the second preparation is selected from the same group as the first preparation. However, the amount of the mucosal adhesive in the second preparation is in the range of 0-200 mg, preferably 0-80 mg, better 0-50 mg.

In some particularly preferred embodiments, wherein the cellulose derivative in the second preparation is selected from the same group as the first preparation. However, the amount of the cellulose derivative in the second preparation is in the range 0-200 mg, preferably 0-100 mg, better 0-50 mg.

In some particularly preferred embodiments, wherein the lipophilic agent is selected from the same group as the first preparation. However, the weight percentage of the lipophilic agent in the second preparation is higher than the one in the first preparation.

In some particularly preferred embodiments, wherein the gargle preparation has one or more characteristics as follows:

1) the percentage weight of the polysaccharide gum or the pharmaceutical gel to the total weight of the first preparation is in the range 0-100%, preferably 50-95%, better 60-80%;

2) the percentage weight of the pharmaceutical acid to the total dry weight of the second preparation is in the range 0-100%;

3) the percentage weight of the adsorbent to the total amount of the second preparation is in the range 0-100%; and

4) the percentage weight of the total amount of the pharmaceutical protein (including enzyme if any) or the polyvinyl pyrrolidone to the total amount of the second preparation is in the range 0-100%.

In some more particularly preferred embodiments, wherein the weight percentage of the maltodextrin in the first preparation is not more than10%, preferably not more than 5%, better not more than 3%.

In some particularly preferred embodiments, wherein the weight percentage of the carrageenan content in the first preparation is not more than 50%, preferably not more than 30%, better not more than 20%.

In some more particularly preferred embodiments, wherein the second preparation comprises pharmaceutical acid, adsorbent, pharmaceutical protein and enzyme, oil-water emulsion, or any combination thereof.

In some particularly preferred embodiments, wherein the weight percentage of the pharmaceutical acid in the second preparation is in the range 0-30% when the second preparation only contains the aqueous solution of the pharmaceutical acid.

In some more particularly preferred embodiments, wherein the weight percentage of the pharmaceutical acid in the second preparation is in the range 2-7%, preferably 2-6%, better 2-5% with a pH value in the range 2.8-7 or 6-9 when the second preparation only contains the aqueous solution of the pharmaceutical acid.

In some particularly preferred embodiments, wherein the weight percentage of the adsorbent in the second preparation is in the range 60-100%, preferably 80-95%.

In some particularly preferred embodiments, wherein the weight percentage of the total amount of the pharmaceutical protein and enzyme in the gargle preparation or the weight percentage of the polyvinyl pyrrolidone to the total amount of the second preparation is in the range 70-100%, preferably 80-95%.

In some particularly preferred embodiments, wherein the gargle preparation comprises excipients that are selected from the group of pharmaceutical pH adjusting agent, surfactant, effervescent agent, dispersing agent, suspending agent, emulsifying agent, flavoring agent, fragrances, gel matrix, essential oil, oral freshener, preservatives, or any combination thereof.

In some more particularly preferred embodiments, wherein the pH adjusting agent is selected from the group if malic acid, tartaric acid, citric acid, fumaric acid, lactic acid, acetic acid, gluconic acid, maleic acid, succinic acid, hydrochloric acid, phosphoric acid, the salts of these acids, and any combination thereof.

In some more particularly preferred embodiments, wherein the effervescent agent is selected from the group of sodium bicarbonate, baking soda, citric acid, tartaric acid, and any combination thereof.

In some more particularly preferred embodiments, wherein the dispersing agent is selected from the group of mannitol, xylitol, sugar, disaccharide, mono-saccharide, cellulose derivatives, pharmaceutical polymeric materials, and any combination thereof.

In some more particularly preferred embodiments, wherein the flavoring agent is selected from the group of sweetener, sodium chloride, other taste agents, and any combination thereof.

In some more particularly preferred embodiments, wherein the suspending agent is selected from the group of surfactant, biological adhesive, polysaccharide gum, cellulose derivative, pharmaceutical polymeric material, and any combination thereof.

In some more particularly preferred embodiments, wherein the pH value in both the first preparation and the second preparation is in the range 2-7.5. Preferably, the pH value of the first preparation is in the range 4-7 and the pH value of the second preparation is in the range 3-7 or 6-9.

In some particularly preferred embodiments, wherein the gargle preparation is spat out after use.

In some particularly preferred embodiments, wherein the gargle preparation is used for gargling for 0.2-10 minutes, preferably 1-8 minutes, better 2-5 minutes.

In some particularly preferred embodiments, wherein the gargle preparation is provided in a dosage form of powder, capsule, rapidly disintegrating tablet, solution, suspension, emulsion, liquid spray, syrup, ointment, cream, pharmaceutical gel base for oral use, chewing gum, chewing candy, or oral adhesive patch.

In some particularly preferred embodiments, wherein the first preparation is used within 0-3 minutes before taking an oral medicine or food and the second preparation is used within 0-5 minutes after the oral medicine or food is taken.

In some more particularly preferred embodiments, wherein the first preparation is used within 0-2 minutes, preferably 0-0.2 minute, before taking an oral medicine or food.

In some more particularly preferred embodiments, wherein the second preparation is used within 0-2 minutes, preferably 0-0.2 minute, after an oral medicine or food is taken.

In some particularly preferred embodiments, wherein the gargle preparation is able to reduce 30-40% of the feeling of the bitter taste in an oral medicine or food as compared to the feeling of others who do not use the gargle preparation.

In some more particularly preferred embodiments, wherein the gargle preparation is able to reduce the feeling of the bitter taste in an oral medicine or food for at least 50%, preferably 60%, more preferably 70%, better 80%, even better 90%, the best 95%.

In some more particularly preferred embodiments, wherein the gargle preparation is able to improve symptoms of dulled taste, jaded palates, or appetite losing.

The present invention provides a method for bitter taste removal, preferably comprising the at least one of following steps:

1) gargle with the first preparation right before taking an oral medicine or food;

2) gargle with the second preparation right after taking the oral medicine or food.

The current invention provides a product that comprises the first and second packages. The first package contains the first preparation of the gargle preparation and the second package contains the second preparation of the gargle preparation. When the first package and the second package can be distributed, sold, and used together, they can also be done so separately and independently for the purpose of bitter taste removal.

The foregoing description of preferred embodiments for the gargle preparation and the method for bitter taste removal is presented for the purposes of summary. Only preferred embodiments are discussed. They are not intended to be exhaustive or to limit the invention to the precise form disclosed. The features and characteristics as discussed for some embodiments may also be applied to other embodiments in different means, which are not fully described one by one here but fell within the scope of current invention.

### BRIEF DESCRIPTION OF THE DRAWINGS

FIG. 1-1 illustrates the intensity of bitter sensing profile of radix sophorae flavescentis. The area under each curve represents the total amount of bitter sensing when radix sophorae flavescentis is taken with or without the gargling treatment.

FIG. 1-2 illustrates the intensity of bitter sensing profile of radix sophorae flavescentis during the first six minutes.

FIG. 2 illustrates the membrane dialysis profile for the decoction of Radix sophorae flavescentis, which is one of experimental results tested for embodiment 6-2.

FIG. 3 illustrates the membrane dialysis profile for the decoction of Gentiana scabra Bunge, which is one of experimental results tested for embodiment 6-2.

FIG. 4 illustrates the membrane dialysis profile for the decoction of Berberine, which is one of experimental results tested for embodiment 6-2.

FIG. 5 illustrates the membrane dialysis profile for the decoction of Scutellaria baicalensis Georgi, which is one of experimental results tested for embodiment 6-2.

### DETAILED DESCRIPTION OF THE PREFERRED EMBODIMENTS

In a long term research, the inventors unexpectedly obtained gargle formulations that can remove bitter taste. The gargle is simple to prepare and easy to use. It is composed of safe and low-cost ingredients. It can efficiently remove a variety of abnormal tastes associated with current pharmaceutical drugs or traditional Chinese herbal medicines. It has no intervention to the active ingredients of the medicines, so there's no need to alter the drug existing formulation to avoid the unpleasant tastes.

As used herein, all the terms as “bitter drug”, “bitter medicine”, “bitter ingredients”, “bitter food” are equivalent to bitter substances in oral cavity, and can be used interchangeably. As used herein, all the terms such as “bitter”, “bitterness”, “pungent”, “hot-spicy”, “peculiar taste”, “strange taste”, “odd taste”, “unusual taste”, “jaded palates”, dulled taste causing appetite losing, as well as “helitosis” are equivalent to bitter taste or an abnormal and unpleasant taste in oral cavity. Among these abnormal tastes, the bitter taste was the most common one.

As used herein, the term “bitter strength” and “bitter intensity as induced on the tongue” are equivalent each other, and can be used interchangeably. It refers to all the abnormal physiological effects induced in the receptors inside the taste buds when the drug-bud associating occurs.

In a study on the bitterness of protein hydrolysis products, the inventors unexpectedly found a universal channel to dispel the bitter taste. Through an in-depth analysis and research, it was found that not only the bitter taste, but also the odd-strange taste, pungent taste etc., as well as the dulled taste could be removed or improved.

From the perspective of modern pharmaceutical science and technology, bitter taste buds on the tongue are a type of receptors which can initiate bitter taste pulse when they are in associated state or activated state. Just like when specific receptors in the body, associate with the active ingredients in medicines, the desired physiological effects are induced. The bitter ingredients in Chinese herbal medicines, such as alkaloids and glycosides etc., when associate with the bitter taste receptors on the tongue, a similar biological effect are induced. However the effect is named as a “bitter sensing” or “bitter taste”.

From the pharmacokinetics and the concept of pharmacodynamics stand points, if we can reduce the association between the drug bitter components and the bitter taste receptors on the tongue, we should be able to minimize the bitterness to some extent, and to improve the administration of, e.g. the traditional Chinese medicine. The taste bud receptors in the oral cavity are an open system. In this experiment, it was found that there was indeed a biochemically operating space for human to intervene the association between the taste bud receptors and the bitter ingredients.

Therefore, different from those traditional bitter-dispelling channels, such as changing drug dosage forms or altering their excipient components, the inventors found that a timely intervention and altering of the chemical environment in the oral cavity can be developed into a simple, effective, and universal channel to dispel the bitter.

Specifically, a conclusion is drawn from the observations in bitter experiments: the bitter-taste of a medicine is measured by the total amount (or total volume) of bitter-feeling, which corresponds to the total integration of bitter-sensing intensity (or bitter intensity) along the total bitter-sensing time period. Reducing the bitter-sensing intensity and shorten the bitter-sensing time period will reduce the total amount of the bitter feeling, and thus dispelling the bitter taste of a medicine can be achieved.

The following measurements were taken in the process of taking a bitter medicine. The interaction between the taste buds on the tongue and the bitter ingredients in the medicine was analyzed. Free bitter ingredients in medicine+free bitter taste buds on the tongue→→medicine·bud association k1

d[associated medicine·bud]/dt=K₁[free bitter ingredients][free bitter taste buds on the tongue] (1)

Bitter intensity ∝ k [associated medicine·bud]×association area (2a)
∝ Symbols represent “proportion to”, the bracket [ ] refers to concentration or the concentration based on surface area.
At time t, the bitter sensing intensity (or bitter intensity) is symbolized as [B]ₜ:
[B]ₜ=bitter intensity at time t.

[B]ₜ≈k [associated medicine·bud]×association area (2b)

During the process of oral medication, the “free bitter ingredients in the medicine” and the “free bitter taste buds on the tongue” rapidly associate with each other. Hence, the surface concentration of “associated medicine·bud”, namely the term [associated medicine·bud], surges to a high value. So that the induced bitter intensity rises instantaneously, to a maximum value and dominates the medication process.

After swallowing the drug, although the remaining residual drug liquid in the oral cavity is a little, it is still excessive around bitter taste buds. Hence, the bitter taste buds are still saturated by the remaining drug, and then the “associated medicine·bud” start to dissociate slowly. This natural slow dissociation process dominates the after-swallowing time period. As the [associated medicine·bud] concentration decreases, the bitter-sensing intensity is gradually reduced and disappears at the end. Usually the natural dissociation processes goes slowly:

Associated medicine·bud→free bitter ingredients+free bitter taste buds on tongue k₂ d[associated medicine·bud]/dt=(−1)k₂[associated medicine·bud] (3)
K₂>0, it is a first order reaction.

After taking the medicine and before the completely dissociation of the [associated medicine·bud], the bitter-feeling intensity still persists. So the total amount of bitter-sensing should be the integration of bitter-sensing intensity along the time period:

Total amount of bitter-sensing=∫_{0→∞}[bitter-sensing intensity]ₜdt=AUCₜₒₜₐₗ (4)
[Bt]ₜₒₜₐₗ=∫_{0→∞}[B]ₜdt=bitter taste of a medicine≈AUCₜₒₜₐₗ
d[Bt]=[B]ₜdt
[B]ₜdt refers to the bitter-sensing during the time dt.

dt is the instantaneous time snippet δt at the individual bitter-feeling intensity [B]ₜ. Namely, the [associated medicine·bud] remains an instantaneous constant during short time period of δt.

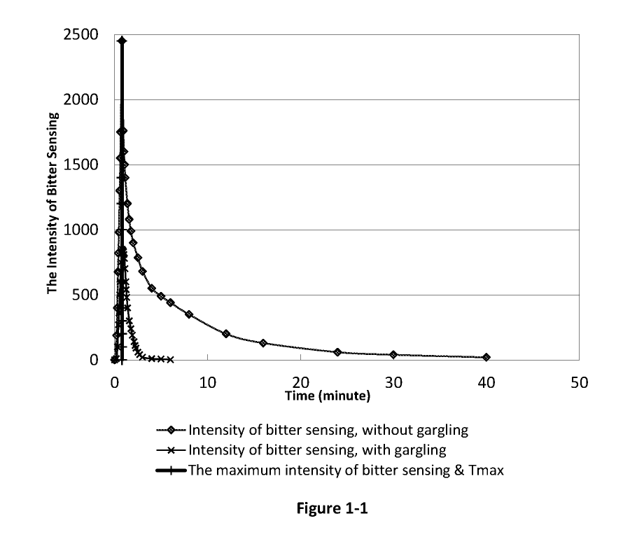

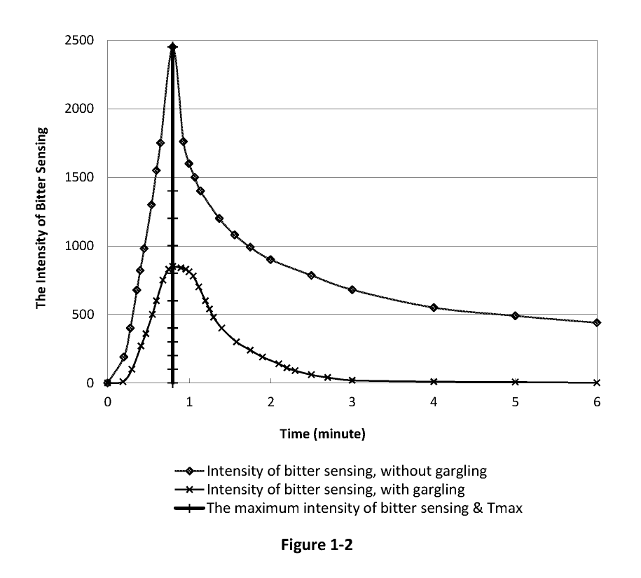

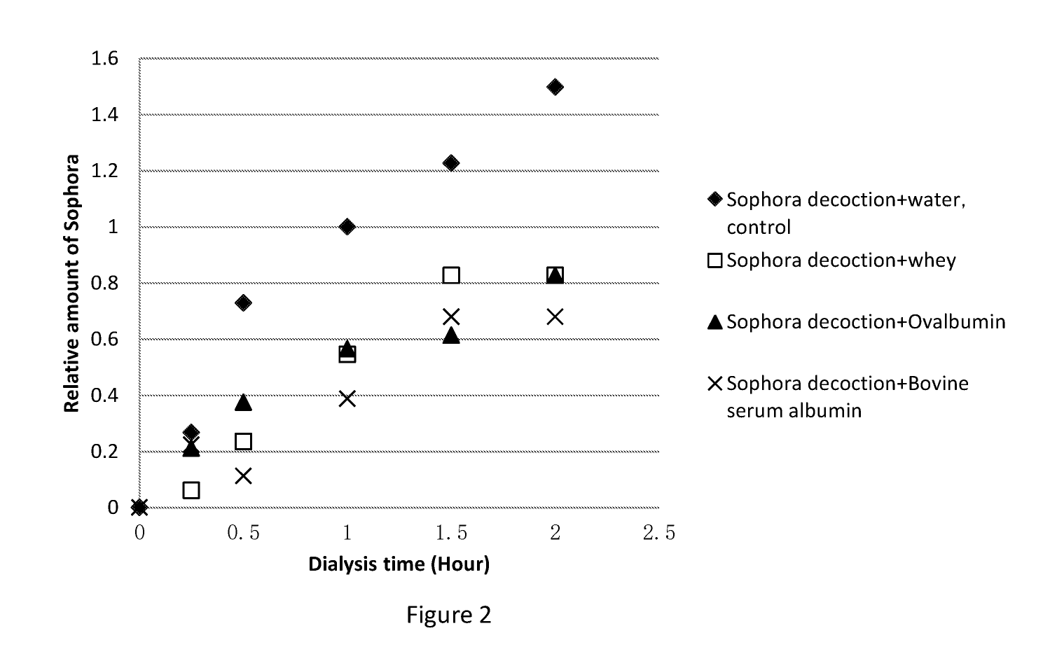

AUCₜₒₜₐₗ refers to the total area under the curve, namely under the bitter-timing profile curve. The corresponding area under the curve is AUC, also see FIGS. 1-1 and 1-2.

[Bt]ₜₒₜₐₗ refers to the total bitter-sensing from time zero (starting to take the medicine)to the end point (bitter sensing vanished). It correctly describes the general feeling of a bitter taste caused by a medicine intake.

Traditionally, instead of the total amount of bitter sensing [Bt]ₜₒₜₐₗ, people used bitter intensity [B]ₜ or the maximum bitter intensity [B]ₘₐₓ (see below) to describe the general bitter sensing of a medicine. The time factor t, namely how long the bitter-sensing lasted contributed a lot to the total bitter taste of the medicine, namely the large % bitter share, was ignored. This was not accurate.

Generally, drug-receptor interaction and its physiological effects are often indirectly reflected in the pharmacokinetic analysis. From the above analysis, the interaction between bitter ingredients and bitter taste buds (the receptors) on the tongue, the related association/dissociation and attenuation, and their overall effects and kinetics, are similar to pharmacokinetics models. For example, they are alike the process of drug absorption, metabolism-attenuation, and elimination.

Therefore several analysis tools in pharmacokinetics can be applied to the analysis of drug inducing bitter taste, such as the drug concentration-time profile curve, the area under the concentration-time profile curve (AUC), and the maximum plasma concentration (C_{Max}) and etc.

The similarities of the two are listed below:
a) The absorption process of a drug corresponds to the association process between the bitter ingredients in medicine and the bitter taste receptors on the tongue;
b) The general drug attenuation process corresponds to the dissociation process of the associated bitter ingredient·buds on the tongue;
c) The bitter-sensing intensity [B]ₜ is equivalent or corresponding to the plasma concentration C of the drug (drug distribution volume in the body is assumed a constant);
d) The maximum bitter-sensing intensity [B]ₘₐₓ corresponds to the maximum plasma concentration C_{Max};
e) The time point for the maximum [B]ₘₐₓ corresponds to the t_{Max} in drug plasma concentration-time profile curve.
f) The half time t½ for the drug to metabolize and attenuate corresponds to the time duration of the associated [bitter ingredients·bud receptors] to dissociate to the half;
g) The bitter intensity-time profile curve, i.e. the curve of [B]ₜ versus time t, is equivalent or corresponding to the drug plasma concentration-time profile curve;
h) The integrated area AUC_{0→∞} from drug-time profile curve, which measures the total biological effects of the intaking medicine, corresponds to the integrated area of [Bt]_{0→∞}≈AUC_{0→∞} from the bitter-time profile curve, which measures the total biological effects as “bitter taste of a drug”, namely the whole amount of the bitter-sensing induced by a medicine.

If the maximum bitter intensity [B]ₘₐₓ and its time t=T_{Max} is set as a time point to divide the bitter-time profile curve into two parts, the total amount of bitter sensing [Bt]ₜₒₜₐₗ=AUCₜₒₜₐₗ can be ascribed to result from two contributions; the first contribution of bitter sensing directly comes from a medication process at t [symbol] ₘₐₓ, [Bt]_{0→t}≈AUC_{0→t}. The second contribution of bitter sensing comes in the retained period t>t_{Max} (i.e. after the medication done) [Bt]_{t→∞}≈AUC_{t→∞}.

Hence the total bitter taste [Bt]_{0→∞}≈AUC_{0→∞} consists of two parts:

AUCₜₒₜₐₗ=AUC_{0→t}+AUC_{t→∞}≈[Bt]_{0→t}+[Bt]_{0→∞}=[Bt]_{0→∞}=[Bt]ₜₒₜₐₗ

The amount of bitter sensing induced in the medication process

=∫_{0→t}[B]ₜdt=[Bt]_{0→t}≈AUC_{0→t} (5A)

The amount of bitter sensing induced and retained after the medication

=∫_{t→∞}[B]ₜ dt=[Bt]_{t→∞}≈AUC_{t-∞} (5B)

Percentage % contribution of the bitter taste during medication process is:

%=[Bt]_{0→t}/[Bt]ₜₒₜₐₗ×100(%)≈AUC_{0→t}/AUCₜₒₜₐₗ×100 (%)

Percentage % contribution of the bitter taste retained after the medication done is:

%=[Bt]_{t→∞}/[Bt]ₜₒₜₐₗ×100(%)≈AUC_{t→∞/}/AUCₜₒₜₐₗ×100 (%)

The former bitter contribution, as described in (5A), usually is an intrinsic bitter feeling inherent in medicine or some traditional/alternative medicine. In a medication process, if the taste buds on tongue are not protected at all and are fully exposed to the bitter ingredients in the medicine, the drug-induced bitter tasting seems inevitably come with the medication.

While the latter bitter contribution, as described in (5B), is basically independent of the drug efficacy, and not a necessary or inevitable bitter taste. However, its contribution to the total bitter-feeling is essential and is not negligible.

If a particular efficacy of a particular medicine, or a portion of its efficacy, is owing to the bitter stimulus (i.e. like certain “taste acupuncture”) to the buds on tongue, then this medicine will not be included in the scope of this bitter dispelling discussed.

The later research and development demonstrated that by using the invented bitter dispelling preparations and their application methods, the bitter intensity and the retaining time were significantly decreased and shortened respectively. As expected, the total bitter-sensing amount or the bitter taste of the medicine tested dramatically reduced or even eliminated. It was also expected that the area under the bitter intensity-time profile curve was largely shrunk.

The current methods of dispelling bitter taste during medication, either physical encapsulation or chemical complexation, focus on alternating the environment of the medicine. This invention uses the techniques to encapsulate the taste buds and alter the environment of around them. It can be used in wider range of applications.

Bitterness and other unpleasant tastes of medicines, or the side effects of medicine that cause temporary loss of taste or appetite, are due to the medicine directly touch the surface of the taste buds and oral mucosa. In this invention, the inventor created formulas from pharmaceutical adjuvant and food additives, use them as a barrier to block the receptors in the taste buds and oral mucosa, hence dispel the bitter taste of the medicine.

Keeping the taste buds from being touch with the bad tasting medicine is the ultimate way to avoid the unpleasant tastes.

Namely, (1)the newly invented technique can create a temporary thin layer of barrier on the taste buds and oral mucosa, to protect the full or partial area of the taste buds against the direct attack of bitter ingredients and other side effect factors in a specific time. (2) the newly invented technique can create a temporary chemical environment, which improves the dissociation between the taste buds and the remaining medicines, and increases the elution rate of the bitter ingredients from the oral cavity.

For pre-medication protection, this invention uses an invented gargle formulations to rinse and coat the oral surface. After spitting the gargle out, the remaining gargle left on the oral surface and formed a loose thin layer of adhesive film to cover the tongue and mucosa. During the immediate medication, the thin layer film can mask the taste buds and the oral mucosa to protect the receptor in the taste buds from associating with and activating by the bitter ingredients and other side effect factors. Hence reduce the bitterness.

By increasing the uncovered taste buds (1) and reducing the medicine/taste buds contact area (2), the feeling of the bitterness is reduced.

The invented gargle for pre-medication use, are made from a selection of drug/food grade proteins, lipophilic components, adhesives for bio-mucosa, polysaccharide viscous gums, as well as pharmaceutical inert ingredients such as Hydrogel and oily gel base. The ingredients of the gargle are inert and the amount is the minimum, they would not have significant impact on the effectiveness of medication. The layer left from the gargle acts as a barrier over the taste buds, and prevent them from being directly attacked by the bitter ingredients in the medicine. Another form of the barrier could be a gel based oral medicine patch with no pharmaceutical active ingredient, it can be used to cover the desired taste buds area before medication. It could also combine with the pre-medication and post-medication gargle preparations to further dispel bitter taste.

Normally the natural dissociation of the [associated medicine·bud] after medication is slow. The related bitter-retention trials proved the reasoning in equ.5B. The bitterness left post medication is not necessarily part of the efficacy of the medicine.

Radix Sophorae Flavescent decoction as a common traditional Chinese medicine is extremely bitter during medication (high bitter intensity and a large area AUC_{0→t} under the curve during medication). After medication it stubbornly associates with the taste buds and adheres on the oral mucosa and pharyngeal mucosa. The [medicine·buds] association slowly unbound after taking the medicine, and the bitterness lasts for a long time (corresponding to the area of AUC_{t→∞}). Therefore, the amount of bitter taste (large area of AUC_{0→t}) in the post-medication lingering (large area of AUC_{t→∞}), and thus their summation, namely the total area under the curve AUCₜₒₜₐₗ=AUC_{0→t}+AUC_{t→∞}, are very large. The inventor used Radix Sophora flavescens (from province Hebei, PRC), gentian (from province Heilongjiang), Sichuan rhizoma coptidis (from Sichuan province), and cortex phellodendri to do human trial testing. By using the above decoction as a gargle solution respectively, recorded the time and the feeling of bitter intensity, then spit the decoction and gargled with water, continued to observe and record the retained bitter sensing versus the retention time until the bitter feeling disappeared. All of these medicines were proved of strong bitter taste, among them Sophora flavescens showed the strongest bitter intensity and longest bitter retention time.

The invention also found that, applying an effective solid or liquid gargle preparation immediately after medication in oral cavity to catalyze the dissociation or artificially damaged the [medicine·bud receptors] association, speeds up the chemical balance to shift to disintegration, and the half-life t½ of the [medicine·bud receptors] association can be minimized to almost negligible. The invented post-medication gargle technology for dispelling bitter includes:

1) Instantly change the pH value in oral cavity, such as allowing the oral pH to be in a momentary acidic condition. The hydrogen ions will alter the polarity of the bitter taste alkaloid molecules left from the medicine, and break the bond between the alkaloid and the receptor on the tongue.

2) Gargle made from edible adsorbents or lipophilic excipients, such as: activated carbon, activated bamboo charcoal powder, macro porous resin (e.g. amberlyst 15 series), cellulose derivatives, ethanol, glycerol, surfactants (lecithin and other phospholipids), edible oil-water emulsion, etc. The adsorbents will strongly adsorb and elute the bitter ingredients in the residual medicine. The lipophilic excipient will shift the partition coefficient of the bitter ingredients from the oral mucosa to the solution side. Surfactants and lecithin may encapsulate the bitter ingredients and/or block the taste buds from touching the residue medicine.

These measures will impel the bitter ingredients to dissociate more quickly from the taste buds receptor, and elute the residual medicine from oral cavity faster than not using the gargle.

3) Gargle made from enzymes and hydrophobic proteins. The former can be, for example α-, β-glycosidase, Poly-glycosides, xylanase, amylase, cellulase, hemicellulose, rhamnosidase, galactosidase, Lactose enzymes etc. The later can be, for example, whey protein, sodium caseinate, milk powder, various albumins; or polyvinyl pyrrolidone (PVP/PPVP). The formulation has strong complexing power, which will compete with the taste bud receptors to associate with the alkaloids and glycosides etc. in herbal medicine. The cellulase can also absorb the medicine ingredients. The bitter ingredients are thus unbound and removed from the receptors on the taste buds

The above formulation/methods could also be combined and developed to target specific herbal medicines or patient groups. For example, adding tannic acid to the post-medication formula to precipitate alkaloid that causes bitterness. Another example is using patches that are coated with pharmaceutical grade adhesive gum/polymer, or edible oil gel/hydrogel, combine with the above formula, to temporarily cover the taste buds area on the tongue. The formula can also be developed as oral powder or spray for ease to use by children

In General, all of these pre- and post-medication formulations, without changing the prescription of herbal medicine, will substantially reduce the area AUC_{0→∞} under the curve, remove 50-80% of the bitter taste, greatly help making the taste in the mouth quickly return to normal.

Another positive finding in the human trial of the invention is, the gargle formulas not only help minimize the bitter taste in pre and post medication, they also reduce the chances of losing appetites after taking the bitter herbal medicines. This could be due to the protection on the taste buds, which keeps the taste system intact when having medication.

The pre-medication formulation is a gargle preparation that is used before taking the medicine. Take a small sip of gargle, swish it in the mouth for a few seconds, and spit it out. Take the medicine right after that. The gargle contains drug edible proteins, lipophilic excipients, adhesives for bio-mucosa, polysaccharide viscous gums, pharmaceutical grade hydrogel or oil gel base, protein mixture of adsorbents, and optional excipients such as lecithin, sweet masking, oral freshener, etc.

After the gargle is used, a loose protective film is formed on the surface of oral mucosa and the taste buds. This thin layer of film protects the taste buds, which would otherwise be totally naked and exposed, from the direct attack by the bitter ingredients in the medicine right following the gargling. Thereby it significantly reduces the bitter intensity and the total amount of bitter sensing, AUC_{0→t}, during the medication.

Typically, the main components and the pre-medication mouthwash formulation are selected from the following pharmaceutical grade ingredients: polysaccharide viscous gums, proteins, xanthan gum, carrageenan, guar gum, sodium alginate, arabic gum, gummi tragacanthae, pectin, sophora bean gum, locust bean gum, konjac gum, gellan gum, curdlan, tamarind seed gum, agar, thermal gel, cyclodextrin, maltodextrin of low DE value, starch, cellulose derivatives, CMC, chitosan, whey, milk powder, sodium caseinate, gelatin solution, water-oil emulsion, adsorbents, hydrogel or oil gel, pH adjusting agent, surfactant, lecithin, effervescent agent, dispersing agent, essential oil, oral freshener, preservatives, sweetener and flavor.

These ingredients can be used separately, combined with different amount to form different formulations.

Alternatively, the post-medication preparation of the gargle can be used to dispel the bitter taste after medication. Particularly, rinsing the mouth with the gargle immediately after taking the medicine, it helps the mouth return to normal taste quickly.

This gargle preparation is developed based on the fact that medicine liquid with bitter taste takes a long time to dissociate from the taste buds. In other words, at t=T_{Max}→T_{∞} time period, the amount of bitter feeling AUC_{t→∞} still has a substantial contribution to the total amount of bitter feeling AUCₜₒₜₐₗ. However, this bitter feeling of AUC_{t→∞} in post-medication has nothing to do with the efficacy of the medication.

Typically for the post-medication gargle, one formulation can be a simple acidic elution solution composed of food grade acids. It can immediately change the pH in oral cavity, wash off common drug ingredients that are causing bitter and peculiar taste; another formulation can select from a wide range of adsorbents such as active carbon (including activated bamboo carbon), and macro-porous resin etc. It can completely adsorb and wash off bitter drug ingredients; Other formulations can be a gargle composed of lipophilic elution agents, specific enzymes and proteins, or polyvinyl pyrrolidone series. These ingredients compete with the taste bud receptors for the drug ingredients and hereby break down the medicine-bud association. In addition to those, some acid-base agents, adhesion agents, surfactants, effervescent agent, suspending agent, essential oil, oral freshener, preservatives, sweetener, artificial flavors can also be added to form composite gargle preparation.

The application of the post-medication gargle preparation can break down and elute the residual drug molecules associated with the taste buds, thus completely get rid of the unpleasant taste left after taking herbal medicine.

According to the research in this invention, the pre- or post-medication gargling can remove off 50-90% of the bitterness. Namely, in the bitter intensity-time profile, the area under the curve, AUCₜₒₜₐₗ, will decrease up to 50-80%, comparing to having the medicine without the using the gargle preparation The invention established a pharmacokinetics model of the drug induced bitter feeling, and the bitter-time curve, i.e. a profile for the related bitter intensity versus time. The area under the bitter-time curve, AUC_{0→∞}, corresponds to the total amount of bitter feeling caused by a medication.

Based on this new theories, experimental explorations, and human trials, a new approach and new preparations are invented to remove or avoid bitter or peculiar taste usually caused by medications. Namely before and/or after medication a dedicated set of gargle preparation has to be individually used. The term medicine and/or medication mentioned above, which causes bitter taste in oral cavity, is a collective term. They includes oral pharmaceutical products, herbal or animal products, traditional Chinese medicine, or food supplements, etc. which can change normal taste to bitter or other unpleasant tastes.

The formulation of the two gargle preparations can be developed in the following dosage forms—powder, oral rapid disintegrating tablets, capsules, liquids, emulsions, suspensions, syrups, solution and powder sprays, ointments, creams, oral gel, appropriate medical adhesive patch for oral cavity use, chewing gum, chewing candies, etc. The components of the dosage forms are pharmaceutical grade proteins, enzymes, lipophilic excipients, edible oils, adhesives for bio-mucosa, viscous polysaccharide gum, cellulose derivatives, medicinal hydrogel/oil gel, acids/alkalis, activated carbon, macro-porous resin etc. adsorption agents, surfactants, and the appropriate excipients, respectively.

Main components of the above-mentioned gargle preparation before taking the medicine include pharmaceutical grad protein, adhesion agent, viscous polysaccharides gum, oral gel, oil-water emulsion, cellulose derivatives, activated carbon or macro-porous resin adsorbents, lipophilic agents. They can be an individual component for us, but it is better for them to be formulated with each other. Besides, excipients of acid base regulation agents, surfactants, effervescent agents, dispersing agents, essential oil, oral freshener, preservatives, a small amount of sweetener, flavorings, fragrance and other general excipients are also included.

The protein mentioned above include a formulated whey protein, milk powder, isolated soy protein, sodium casienate, gelatin, albumins and viscous polysaccharide gums, about 100-400 mg for each gargle. When gelatin solution of 2-15% gel is used, warm it into a liquid in a warm bath before use and 1-5 mL for each gargle.

The viscous polysaccharide gums and their formulations include gums, which will form gel or swell in 37° C. water, such as: guar gum, xanthan gum, carrageenan, Arabic gum, tragacanth, sodium alginate, and sophora bean gum, gellan gum, curdlan, tamarind seed gum, konjac gum, agar, pectin, and low DE value maltodextrin. In these polysaccharide gum related formulations, the component range is: most of the gums in the range of 100-500 mg or more, maltodextrin of low DE value about 100-800 mg or more, or an appropriate amount of their gels.

The adhesive agents for bio-mucosa includes Chitosan in the range of about 20-80 mg, Cyclodextrin, β-Cyclodextrin and so on about 50-400 mg.

The cellulose derivative includes carboxymethyl cellulose CMC, hydroxy propyl methyl cellulose with thermal gel properties, methyl-, ethyl-, and hydroxypropyl-cellulose. CMC is about 5-30 mg, the amount of other cellulose derivative about 30-200 mg.

The oil-water emulsion can be: cooking oil with possible essential oil mixed with water, oil accounted for 30-45% (w/w), plus 0.5% CMC or 2-10% Tween 80 or other surfactants like 0.1-1% soya lecithin, poloaxamer etc. In each gargle about 1-25 mL of the emulsion is used. In this formulation, the oil should be a separate sub-package before use and shake up with the separated water solution. The fresh prepared aqueous emulsion can be used in 1-2 weeks.

The gargle formulation for pre-medication is: protein mixed with polysaccharide gum, and/or, with a little of active adsorbents, protein percentage 50-90%; can also be a xanthan gum compatible with CMC, CMC 10-20%; can also be a combination of xanthan gum, low DE value maltodextrin and CMC, 80%, 15%, and 5% respectively; Guar or a combination of guar gum, low DE value maltodextrin and CMC, 80%, 15%, and 5% respectively.

The pH regulator, effervescent agent, dispersing agent used in the invention are selected from the edible acids and bases and their salts, e.g. baking soda, xylitol, mannitol, cellulose derivative, and so on. Also there includes some oral freshener and flavorings such as sweeteners and flavors.

The gargle preparation for post-medication use includes the following ingredients: edible acids, enzymes, proteins, polyvinyl pyrrolidone series, biological mucosal adhesive agent, cellulose derivatives, oil-water emulsion, activated carbon and activated bamboo charcoal, macro porous resin, other adsorption agent. They can be individual component used, or be formulated with each other. There also includes drug edible pH regulators, effervescent agent, dispersant, surfactant, a small amount of sweetener, flavors, fragrance, Flavorings, essential oil, oral freshener, preservatives, and other general excipients.

The acids include malic acid, tartaric acid, citric acid, lactic acid, fumaric acid, acetic acid, gluconic acid, hydrochloric acid, phosphoric acid, and their salts. They can be a single acid or acid-acid formulation, or a formulation with other major excipients.

The concentration of the acid content is 2-7%, with pH 2.8-7, or 6-9, combined with sweetener, flavorings, and/or, a certain amount of bio-mucosa adhesive agent, lipophilic excipients such as glycerin, polyethylene glycol, alcohol, fragrance and other general excipients.

The dosage forms include a gargle preparation, or a powder-liquid spray, or those can be transformed into a mouth washing solution such as: acid powder, oral rapidly disintegrating tablet, gel, chewing gum, candy etc.

Different acid-acid combinations can create different tastes, e.g. malic acid+tartaric acid about 3:1, or Malic acid+lactic acid about 3:1 or 1;3, or citric acid+lactic acid about 3:1 or 1:3, or citric acid+tartaric acid, about 3:1 or 1:3, and so on.

In the protein formulation for post-medication gargle, enzymes and protein are the main components. Enzymes include α-, β-glycosidase, poly-glycosides, amylase, cellulase, hemi-cellulase, xylanase, rhamnosidase, galactosidase, Lactose enzymes. Protein includes whey protein, milk powder, albumin, isolated soy protein, gelatin, collagen, etc. Each dose has 30-200 mg enzyme, 100-500 mg of protein, with 20-100 mg of Chitosan and cellulose derivatives or similar amount of polyvinyl pyrrolidone. Also add proper amount of effervescent agent, dispersing agent, pH adjusting agent, a small amount of sweetener, flavorings, fragrance and other general excipients.

The formulated dosage form includes: fast disintegrating tablet, powder, tablets, capsules, chewing gum, emulsions, suspension containing the powder, oral gel. When the above materials dissolve or partially dissolve in the mouth, the pH value is in the range 4-7 or 6-9.

The emulsion formulation for post-medication gargle includes the oil-water emulsion preparation: cooking oil with possible essential oil, and water, oil accounted for 30-45% (w/w), plus 0.5% CMC or 2-10% Tween 80 and/or other surfactants as 0.1-1% soya lecithin, poloxamer. And add pH adjusting agent, a small amount of sweetener, flavorings, fragrance and other general excipients, with pH 3-7, or 6-9, 1-5 mL per dose. Cooking oil should be separately installed before use, mix and shake up with the aqueous solution on using. The mixed aqueous emulsion can be used for in 1-2 weeks.

The adsorption formulation for post-medication gargle includes activated carbon, and/or, adsorptive macro-porous resin etc. adsorbents as the main ingredients, which may select from the adsorbents such as: activated charcoal, activated bamboo charcoal powder, macro porous resin, zeolites, magnesium silicate, diatomaceous earth, calcium silicate, aluminum silicate, silicone, chemically modified silicone, and protein as well as polyvinyl pyrrolidone series, Chitosan and cellulose derivatives. Each dose contains 100-400 mg of adsorbent, 0-100% of activated carbon, and also contains 20-200 mg of protein, Chitosan and cellulose derivative for oral mucosa adhesives. Also includes effervescent agent, dispersing agent, suspending agent, pH adjusting agent, essential oil, oral freshener, preservatives, a small amount of sweetener, flavorings, fragrance and other general excipients. Its formulation dosage form includes fast disintegrating tablet, powder, tablet, capsules, emulsions, suspension containing the powder, gel for oral cavity etc. The better pH, for example when activated carbon suspends in the mouth, is 3-7 or 6-9 for oral liquid.

The discussion above is only to explain the mechanisms of bitter taste removal in this invention. It does not limit the scope of this invention.

Unlike the prior technology for removing the bitter taste, the products and methods disclosed by the current invention have the following advantages:

(1) The gargle preparation disclosed by the current invention is simple, effective, safe, and inexpensive. It can be applied to oral medicines or foods with the bitter taste regardless their ingredients.

(2) Because there is no need for taking extra medicine when applying the current invention, there is no interference to the oral medicine being taken by the user. In addition, there is no pharmacological effect on the user's body due to the inertness of the gargle preparation disclosed by the current invention;

(3) The method disclosed by the current invention is easy to be applied for bitter taste removal;

(4) The gargle preparation and method disclosed by the current invention are effective on bitter taste removal and suitable for almost all oral medicines. Therefore, it is good for the promotion of traditional herbal medicines;

(5) The gargle preparation and method disclosed by the current invention have solved the question for the first time on how to maintain hygiene and healthy status of taste buds in patients taking oral medicine with the bitter taste. They have also solved the issue on temporary loss of taste and appetite for patients who have taken oral medicine with the bitter taste for a long period of time. Patients' life quality is significantly improved due to the current invention.

The current invention is further illustrated by the following embodiments described in detail. It should be understood that these embodiments are for the illustrative purpose only and not to limit the scope of the current invention. Any experimental condition that is not specifically pointed out in the discussion should be interpreted in accordance with the knowledge generally understood and accepted by the scientists or recommended by the manufacturers. Unless stated otherwise, all percentages and portion numbers are weight-based.

Some commonly known plants used in traditional herbal medicine with the bitter taste like sophora, gentian, berberine, phellodendron, coptis, caulis clematidis armandii, gardenia, viola yedoensis makino, real Elm, madder, skullcap, or any combination thereof, are used to evaluate the effect of the gargle preparations on removing the bitter taste.

### EXAMPLE 1

### The Bitter Taste at the Time Taking Medicine, and its Evolution Versus Time of Bitter Feeling After Taking Medicine Began and Correspond to the Time

We were using Sophora flavescens (from province Hebei), gentian (from province Heilongjiang) and coptis (from province Sichuan) decoctions to make several human trials respectively. Provided that in addition to the water served to gargle the mouth, there was no other manual intervention before and after taking decoctions, i.e. no eating of sweets or candy, no gargle treatment such as that in embodiment of 2 to 7. We took about 5-10 mL of the cooled decoctions in mouth and started to record the time and bitter taste feeling, i.e. the first time recording. Spat the decoction out after about a minute, followed by a clean water gargling once, and then started again to continue to observe and record the retention time and the remaining bitter taste (the second time recording), took several water gargling in between. The observed results were:

1) Strongly the bitter taste of Sophora flavescens AIT. had Sophora flavescens AIT decoction in mouth about 1 minutes, recorded the bitter taste and spat it out, followed by a clean water gargling once, and then started a second time observing and recording. Each subsequent 5 min took a water gargling again. Bitter taste retention was observed as: 0-30 min strong bitter taste, 30-45 min obviously bitter, 45-60 min bitter taste weak, >60 min bitter taste disappeared. Generally, the bitter taste decay slowly, with a residue adhesive in the throat, hard to dissociate and last long.

2) Gentian gave strong bitter taste. In the second time recording, strong bitter taste remained to 20 min when spat it and saliva out, followed by a water gargling. In 20-35 min an obvious bitterness still continued to stay. At 35 min spat it and saliva out and took a water gargling again. An acceptable bitterness retention continued about 15 min longer. At 50 min the bitter taste disappeared. A total of approximately 50 min long retention was observed. Generally the bitter taste decayed slowly.

3) Coptis gave strong bitter taste. In the second time recording, the retained bitter taste of coptis chinensis was similar to that of gentian. Coptis seemed to stimulate more saliva, its mixture with saliva with a yellow color was spat at time points of 10 min and 25 min, gargled with water respectively each time. Generally the retained bitter taste continued to and ended at about 40 min. Its bitter taste decayed slowly.

4) real Elm: its decoction tasted astringent and bitter, more astringent than bitter. In the second time recording, found it faster to dissociate on tongue and in mouth, and to stimulate the production of more saliva. Its bitter taste decayed quickly, approximately 5-6 minutes after taking the decoction, the bitter taste disappeared.

5) Gardenia, akebia, and viola yedoensis Makino, madder, and scutellaria, their decoctions tasted slightly bitter with a peculiar, foul taste. Their bitter retention in the second recording time was found not long, it decayed more quickly. Apart from the gardenia, at about 5 min the bitter taste weakened and disappeared. Gardenia retained slightly longer, its bitter taste disappeared at time>5 min.

### EXAMPLE 2-1

### Take a Pre-Medication Gargling, by Using a Special Gargle Preparation Right Before the Medication, can Reduce the Total Amount of Bitter Feeling and Bitter Intensity During the Process of Medication

Our human subject tests found that taking a pre-medication gargle preparation and then spitting it out right before taking a cold decoction of Chinese herbal medicines, could considerably reduce the related bitter intensity and the total amount of bitter feeling.

This is owing to the fact that the taste buds were temporarily covered and blocked by the remaining residual gargle preparation in the pre-medication gargle, which contained viscous polysaccharide gums, oral gel, adhesives for bio-mucosa, oil-water emulsion, cellulose derivative, protein with or without a small amount of adsorbent. The thin layer films thus formed from the residual gargle preparation could temporarily cover and protect the taste buds on the tongue and the mucosa in oral cavity. Thereby it delayed and minimized the taste buds on reaction time to and on the association degree with the bitter ingredients in the medicine. The total amount of the residual adhesive thin layer was small, but enough to momentarily block the taste buds and the oral mucosa.

The components in the gargle preparation were inert, and only a little residue were left in the mouth after gargling and spitting out. In general, the residue would not interfere with the prescriptions of the Chinese or other nation's traditional medicine. Also it was easy to wash and rinse it out from the mouth cavity after finishing the drug dose. In short, as compared to prior method to dispel bitter taste by using a massive masking agent added, the residue in the invented gargle method was very minimal. Typically, the embodiment of gargle preparation for pre-medication use could be selected from the following formulations:

1) Formulation of protein powder, which contained 30-70% (weight percentage, same below) of protein,70-30% of viscous polysaccharides gums, or a warmed gelatin solution (concentration 5-10%). Among them included: casein proteins or sodium caseinate, whey protein, albumins, isolated soy protein, milk powders, collagen etc. Polysaccharide viscous gums included: xanthan gum, guar gum, tragacanth, carrageenan, sodium alginate, arabic gum, pectin, sophora bean gum and konjac gum, low DE value maltodextrin.

2) Protein formulation contained a small amount of adsorbents. It included a little activated carbon, or activated bamboo charcoal powder, macro-porous resin, or other adsorbents, and mixed with protein powder and viscous polysaccharide gum powder respectively; 1-5%, 35%, 60%.

3) Oil-water emulsion formulation, it included the edible vegetable oil 30-45%, water 70-55%, plus 0.5% CMC or lecithin, 0.5-1% polysaccharide gel and/or low DE value such as maltodextrin. Edible vegetable oil should be bottled separately, and be mixed before using with the corresponding water emulsifying. In pre-medication gargling, shake the emulsion once again. The well mixed emulsion could continue to be used in 1-2 weeks. If the lipids were not preferred, other gargle formulations of the invention could be selected instead.

4) Polysaccharide gum formulation, it included xanthan gum, guar gum, sodium alginate, tragacanth, carrageenan, pectin, etc. as partially shown in table 1 below.

TABLE 1

| | % W | | | | | | | | |
|---|---|---|---|---|---|---|---|---|---|
| | | | | | | Sophora | Knojac | | |
| Formulation | Xanthan | | | Sodium | Gum | bean | gum | | |
| # | gum | Guargum | Carrageenan | alginate | Arabic | gum | KGM | Maltodextrin | Pectin |
| X-1 | 100 | | | | | | | | |
| X-2 | 95 | | | | | | | | |
| X-3 | 80 | 10 | | | | | 9 | | |
| X-4 | 80 | | 10 | | | 7 | | | |
| G-1 | | 100 | | | | | | | |
| G-2 | 19 | 80 | | | | | | | |
| G-3 | | 80 | | | | | 10 | | |
| C-1 | | | 100 | | | | | | |
| C-2 | 10 | 5 | 80 | | | 5 | | | |
| C-3 | | | 80 | | | 10 | 5 | | |
| AL-1 | | | | 100 | | | | | |
| AL-2 | | | | 90 | | | 5 | | |
| AL-3 | 5 | 5 | | 85 | | | | | 5 |
| Ar-1 | | | | | 100 | | | | |
| Ar-2 | | | | | 89 | | | | |
| Ar-3 | 5 | 3 | | | 80 | | | | 5 |
| L-1 | | | | | | 100 | | | |
| L-2 | 10 | 5 | 5 | | | 80 | | | |
| L-3 | 5 | 10 | 5 | 5 | | 72 | | | |
| KGM-1 | | | | | | | 100 | | |
| KGM-2 | 10 | | 10 | | | | 80 | | |
| KGM-3 | 15 | | 10 | | | | 70 | 3 | 2 |
| Dex-1 | 5 | | | | | | | 80 | |
| Dex-2 | 20 | 5 | 5 | | | | | 70 | |
| Dex-3 | 20 | 10 | 10 | | | | | 60 | |
| St-1 | 10 | | | | | | | | |
| St-2 | 10 | 5 | | | | 5 | | | |
| St-3 | | 10 | | 10 | | | 10 | | |
| | | % W | | | | | | | shielding |
| | | Other | Water- | | | Water- | | | effect of |
| | Formulation | types | soluble | | | soluble | Acid | Sweeteners/ | bitter taste |
| | # | of gum* | starch | CMC | Chitosan | protein | salt | Flavor | on taste buds |
| | X-1 | | | | | | | | Excellent |
| | X-2 | | 4 | | | | <1 | <1 | Excellent |
| | X-3 | | | | | | <1 | <1 | Excellent |
| | X-4 | | 2 | | | | <1 | <1 | Excellent |
| | G-1 | | | | | | | | Excellent |
| | G-2 | | | | | | <1 | <1 | Excellent |
| | G-3 | | 4 | | | 5 | <1 | <1 | Excellent |
| | C-1 | | | | | | | | Good |
| | C-2 | | | | | | | | Good |
| | C-3 | | | | | 4 | <1 | <1 | Good |
| | AL-1 | | | | | | | | Excellent |
| | AL-2 | | | 5 | 5 | | | | Excellent |
| | AL-3 | | | | | | | <1 | Excellent |
| | Ar-1 | | | | | | | | Excellent |
| | Ar-2 | | | | | 10 | <1 | <1 | Excellent |
| | Ar-3 | | 2 | 3 | 2 | | | | Excellent |
| | L-1 | | | | | | | | Excellent |
| | L-2 | | | | | | | | Excellent |
| | L-3 | 3 | | | | | | | Excellent |
| | KGM-1 | | | | | | | <1 | Excellent |
| | KGM-2 | | | | | | | | Excellent |
| | KGM-3 | | | | | | | | Excellent |
| | Dex-1 | | | 5 | 10 | | | | Poor |
| | Dex-2 | | | | | | | | Poor |
| | Dex-3 | | | | | | | | Poor |
| | St-1 | | 90 | | | | | | Poor |
| | St-2 | | 80 | | | | | | Poor |
| | St-3 | | 70 | | | | | | Poor |

Note that in table 1, “other gums” included tragacanth, agar, gellan gum, tamarind gum and so on. Acid salt refers to food grade acids and their corresponding salt, used to adjust pH, if wanted. In addition, proper amount of xylitol or mannitol powder (about 10%) could be added, in order to disperse the gargle preparation in oral cavity. Being poor means the formulation is not sticky enough and a large dose needs to be used.

Table 1 tests found polysaccharide gums of xanthan gum, guar gum, sodium alginate, tragacanth, Sophora bean gum and konjac gum, Arabic gum and their combination are the best. Before taking bitter medicine, a 100-500 mg (mastered by every individual) of the gargle powder formulation was poured on the tongue, gradually stirred to dissolve and swell, mixed it with saliva. chewed it slowly, while continuously mixing and coating with the tongue, until the tongue, sublingual, and whole oral mucosa were coated with a thin layer of sticky gum or gel. This step would take about a few minutes. Spat out the vast majority of the gum, then immediately took in the warm and cold bitter decoction or medicines in other form.

Owing to the protection from the adhesive thin covering layer, the bitter intensity and the whole bitter feeling were significantly reduced. Found in the test that the bitter taste on the area of tongue surface, area of back part of tongue, and the area of the surface of throat, reduced more than that in other area, where the mucosa was plainer than the other area and the taste buds there were more protected by covers. And the bitter feelings was focused on, or redistributed to, the vertical surfaces on both sides of the tongue. Apparently more friction there, the protective thin layer film was more difficult to set up there.

Also observed in vitro, the strength of the viscous polysaccharide gel in gargle preparation had certain toleration to erosion by a scouring from a small flow of water.

Oral testing also found that just took a single pre-medication gargling and then did the same as embodiment 1, even without the post-medication gargling, the bitter retention time was reduced by almost half of that in embodiment 1. This suggested that pre-medication gargle preparation could be effective in reducing the association between the taste buds and the bitter ingredients in medicine. Above-mentioned gargle powder formulation can also be capsules, or could also add cellulose derivatives to make oral rapidly disintegrating tablets.

The above-mentioned gargle formulations could be used alone, or could also be used in multiple combinations successively, such as a combination of protein powder and a small amount of adsorbents, a combination of protein powders and viscous polysaccharide gum, a combination use of viscous polysaccharide gum and the oil-water emulsions, and so on.

Above-mentioned gargle formulation could also be added the dispersing agents and sodium bicarbonate effervescent agent in order to dissolve and disperse. Other added components can be: pH regulator, a few sweeteners, flavorings, essential oil, oral freshener, preservatives, and/or flavors, such as to adjust texture.

### EXAMPLE 2-2

### Pre-Medication Gargle Formulation, a Liquid Spray or Toothpaste-Like Hydrogel

Above-mentioned embodiment of 2-1 gargle formulations could be prepared in advanced forms. Such as a toothpaste-like past and was encased in a soft special tube for pressing out, or as a liquid solution and was encased in a hand spray bottle,50-250 mL volume. Before use, warm it in water baths, thermal gel in cold bath, making it mobile and spray every 1-2 mL in the mouth. They would form hydrogel and spread in oral cavity. Children's spray bottle 5-20 mL volume, spray 0.5-1 mL for each puncture.

### EXAMPLE 3-1

### Post-Medication Gargle Formulation, a Lipophilic Acid Solution

The post-medication gargle preparation could be a liquid solution. Its main active ingredients were made up of edible acids, lipophilic accessories, plus some oral freshener, flavors and flavoring agents, preservatives, dissolved in water.

Edible acids and their salts were selected from group: Malic acid, citric acid, lactic acid, tartaric acid, acetic acid, gluconic acid, succinic acid, or a combination thereof. It was soluble in water, total solution acid concentration 2-7%. Used Eating baking soda to adjust pH to 2.5-7 or 6-9.

Specifically, the acid solution could be from a single acid, an acid-acid combinations, or a combination of multiple acids, as shown in table 2. Using different combinations of acids could accommodate different patients' taste and texture.

TABLE 2

| | | | | | | | | | | Protein |
|---|---|---|---|---|---|---|---|---|---|---|
| Formu- | Acid % w | | | | | | Adsorbent % w | | | % w |
| lation | Citric | Malic | Tartaric | Lactic | Acetic | Other acids/ | Activated | Macro- | Other | β- |
| # | acid | acid | acid | acid | acid | alkalis/esters | carbon | pore resin | adsorbents | glucosidase |
| C-1 | 5 | | | | | a.a. | | | | |
| C-2 | 3 | 1 | 1 | | | a.a. | | | | |
| C-3 | 2 | 1 | 1 | 1 | | a.a. | | | | |
| M-1 | | 5 | | | | a.a. | | | | |
| M-2 | 1 | 3 | 1 | | | a.a. | | | | |
| M-3 | 1 | 2 | 1 | 1 | | a.a. | | | | |
| T-1 | | | 5 | | | a.a. | | | | |
| T-2 | 1 | 1 | 3 | | | a.a. | | | | |
| T-3 | 1 | 1 | 2 | 1 | | a.a. | | | | |
| L-1 | | | | 5 | | a.a. | | | | |
| L-2 | 1 | 1 | | 3 | | a.a. | | | | |
| L-3 | 1 | 1 | 1 | 2 | | a.a. | | | | |
| A-1 | | 1 | | | 4 | a.a. | | | | |
| A-2 | | 1 | 1 | | 3 | a.a. | | | | |
| A-3 | 1 | 1 | 1 | | 2 | a.a. | | | | |
| AC-1 | | | | | | | 90 | | | |
| AC-2 | | | | | | | 85 | 5 | | |
| AC-3 | | | | | | | 65 | 25 | | |
| AC-4 | | | | | | | 100 | | | |
| MR-1 | | | | | | | | 90 | | |
| MR-2 | | | | | | | 5 | 85 | | |
| MR-3 | | | | | | | 25 | 65 | | |
| MR-4 | | | | | | | | 100 | | |
| Ads | | | | | | | 25 | 25 | 40 | |
| β-1 | | | | | | | | | | 40 |
| β-2 | | | | | | | | | | 20 |
| β-3 | | | | | | | | | | 10 |
| α-1 | | | | | | | | | | |
| α-2 | | | | | | | | | | |
| α-3 | | | | | | | | | | |
| Cel-1 | | a.a. | | | | | | | | |
| Cel-2 | | a.a. | | | | | | | | |
| Cel-3 | | a.a. | | | | | | | | |
| Hem-1 | | a.a. | | | | | | | | |
| Hem-2 | | a.a. | | | | | | | | |
| Hem-3 | | a.a. | | | | | | | | |
| BSA-1 | | | | | | | | | | |
| BSA-2 | | | | | | | | | | |
| BSA-3 | | | | | | | | | | |
| Prot-1 | | a.a. | | | | | | | | |
| Prot-2 | | a.a. | | | | | | | | |
| Prot-3 | | a.a. | | | | | | | | |
| | | | | | | | Other | | | |
| | Formu- | Protein % w | | | | | excipients % w | | | Effect of |
| | lation | α- | | | Bovine | Other | Effervescent | | | Bitter taste |
| | # | glucosidase | Cellulase | Hemicellulase | albumin | proteins | agent | Sweetener | Flavor | removal |
| | C-1 | | | | | | | <1 | <0.2 | Good |
| | C-2 | | | | | | | <1 | <0.2 | Good |
| | C-3 | | | | | | | <1 | <0.2 | Good |
| | M-1 | | | | | | | <1 | <0.2 | Good |
| | M-2 | | | | | | | <1 | <0.2 | Good |
| | M-3 | | | | | | | <1 | <0.2 | Good |
| | T-1 | | | | | | | <1 | <0.2 | Good |
| | T-2 | | | | | | | <1 | <0.2 | Good |
| | T-3 | | | | | | | <1 | <0.2 | Good |
| | L-1 | | | | | | | <1 | <0.2 | Good |
| | L-2 | | | | | | | <1 | <0.2 | Good |
| | L-3 | | | | | | | <1 | <0.2 | Good |
| | A-1 | | | | | | | <1 | <0.2 | Good |
| | A-2 | | | | | | | <1 | <0.2 | Good |
| | A-3 | | | | | | | <1 | <0.2 | Good |
| | AC-1 | | | | | | 9 | <1 | <0.2 | C.R. |
| | AC-2 | | | | | | 9 | <1 | <0.2 | C.R. |
| | AC-3 | | | | | | 9 | <1 | <0.2 | C.R. |
| | AC-4 | | | | | | | | <0.2 | C.R. |
| | MR-1 | | | | | | 9 | <1 | <0.2 | C.R. |
| | MR-2 | | | | | | 9 | <1 | <0.2 | C.R. |
| | MR-3 | | | | | | 9 | <1 | <0.2 | C.R. |
| | MR-4 | | | | | | | | <0.2 | C.R. |
| | Ads | | | | | | 9 | <1 | <0.2 | C.R. |
| | β-1 | | 25 | 25 | | 10 | | <1 | <0.2 | R. |
| | β-2 | | 20 | 20 | | 40 | | <1 | <0.2 | R. |
| | β-3 | | 30 | 30 | | 30 | | <1 | <0.2 | R. |
| | α-1 | 50 | 20 | 19 | | 10 | | <1 | <0.2 | R. |
| | α-2 | 30 | 25 | 24 | | 20 | | <1 | <0.2 | R. |
| | α-3 | 20 | 25 | 24 | | 30 | | <1 | <0.2 | R. |
| | Cel-1 | | 70 | 20 | | 9 | a.a. | <1 | <0.2 | R. |
| | Cel-2 | | 50 | 25 | | 24 | a.a. | <1 | <0.2 | R. |
| | Cel-3 | | 30 | 20 | | 49 | a.a. | <1 | <0.2 | R. |
| | Hem-1 | | 20 | 70 | | 9 | a.a. | <1 | <0.2 | R. |
| | Hem-2 | | 25 | 50 | | 24 | a.a. | <1 | <0.2 | R. |
| | Hem-3 | | 20 | 30 | | 49 | a.a. | <1 | <0.2 | R. |
| | BSA-1 | | 10 | 10 | 70 | 9 | | <1 | <0.2 | R. |
| | BSA-2 | | 20 | 20 | 50 | 9 | | <1 | <0.2 | R. |
| | BSA-3 | | 20 | 20 | 30 | 29 | | <1 | <0.2 | R. |
| | Prot-1 | | 15 | 14 | | 70 | a.a. | <1 | <0.2 | R. |
| | Prot-2 | | 25 | 24 | | 50 | a.a. | <1 | <0.2 | R. |
| | Prot-3 | | 33 | 33 | | 33 | a.a. | <1 | <0.2 | R. |

a.a.: appropriate amout;
C.R.: completely recovered (taste buds);
R.: recovered (taste buds);
the order of effect from good to bad: C.R. > R. > Good

Note: in table 2, the lipophilic and acidic formulation for gargle was an aqueous solution of acids, acid 2-7%, pH 2.5-7, or 6-9, other acids/alkalis may include edible succinic acid, maleic acid, hydrochloric acid, phosphoric acid, sodium hydroxide, sodium carbonate, sodium bicarbonate, and so on. In post-medication, the residue ingredients causing weak bitter and peculiar-foul taste would generally be rinsed out by the lipophilic acid formulation in this step.

Table 2, the adsorbent formulation for gargle included, macro-porous resin or adsorbent resin which included non-polar, intermediate polar, and polar resin, three groups of resin and the related accessories. While the other adsorbents included Chitosan, β-cyclodextrin, medical silicone, chemically modified silicone, magnesium silicate, zeolites, as well as edible polysaccharide gums and oral freshener. The residual medicine ingredients causing strong bitter taste would be completely cleared away by this adsorbent formulation.

Table 2, in the enzyme-protein formulation for gargle, the other proteins included xylanase (rhamnosidase, galactosidase, Lactose enzymes etc can also be used), milk powder, sodium caseinate, whey, isolated soy protein, gelatin, collagen, egg white protein etc. The combination of protein and certain amounts of enzymes could be used to remove the last remaining bitter ingredients, especially those bitter ingredients adhesive on the back part of tongue, or on the part of throat mucosa. It would rinse the whole oral cavity and quickly return it to normal.

Table 2, in the case of solving the “extremely bitter” medicine problems, to apply the lipophilic acid solution formulation, adsorbent formulation, and protein formulation in turn, is the best procedure of dispelling bitter.

Lipophilic accessories were selected from glycerin, alcohol, polyethylene glycol (PEG), total amount 1-25% (glycerol majority).

Optionally, the formulation also contains surfactant as lecithin, polysorbate 80, poloxamer, or C12 alkyl sulfonic acid sodium salt<0.5%.

Flavors available: sodium glutamate, sweetening agent xylitol, mannitol, aspartame (ASPARTME), or saccharin (<0.02%), or sodium chloride, with a total <0.5%. The available formulation of fresh acidic, sweet, and salty is designed to meet different human tastes. Also be appropriate to add a variety of oral freshener, preservatives, and flavors, such as menthol, peppermint oil, fruit fragrances (orange citrus flavor, cherry, pineapple flavor), spearmint, and so on.

In addition, the inventor also explored commercial vinegar without any extra addition in lieu of the acid formulation in table 2. To some extent, it behaved the same as the acid formulation and could immediately wash off the bitter taste in some weak bitter medicine. But vinegar contains quite a lot of volatile aldehydes, ketones, and esters, which may interfere with the effectiveness of medicines taken. In addition, as far as dispelling strong bitter taste, vinegar works not well for the strong bitter medicines, it is not recommended to use it as a gargle agent. Similarly, if not formulated with enzymes and/or with adsorbents and other accessories, a direct application of edible protein powder (such as milk powder, isolated soy protein, whey protein) as a gargle agent for post-medication would not be effective enough. Prior to the vinegar formulation to be adjusted according to this invention, e.g. its pH, its concentration, the lipophilic excipients and oral freshener applied, common vinegar is not recommended as a gargle solution to remove bitter taste.

Implementation examples showed that post-medication of those medicines of weaker bitter taste, when immediately took the lipophilic acid formulation of 5-20 mL gargle solution to rinse oral cavity, the drug's bitter taste/smell was very easy to be immediately eliminated, and the mouth quickly became clear.

Lipophilic acidic gargle preparation can also be formulated in the form of rapid disintegrating tablets, capsule, powder, or candy in order to facilitate carrying. The formulated acid in these solid dosages bears the same amount of acid as corresponding to the dose amount in acidic solution. Embodiment of 3-2, post-medication gargle formulation: lipophilic acid liquid sprays Above-mentioned in embodiment of 3-1, the lipophilic and edible acid solution for gargle allows it to be mounted in a special hand press spray bottle, 50-250 mL volume. Spray every 0.5-1 mL in the mouth. Children's spray bottle 5-20 mL volume, spray every 0.1-0.5 mL of edible sour-sweet, lipophilic solution for gargle.

### EXAMPLE 3-3

### Post-medication Gargle Formulation Containing Tannic Acid

Tannins and alkaloids or glycosides will produce co-precipitation. But Tannins is easy to be oxidized and has strong astringent, it should be packaged separately for some patients to select as a particular optional use: (a) 20% of tannic acid glycerol solution, 10 times dilution or more before using, for example 10 mL of the solution add 100 mL of those gargle solutions in embodiments of 3-1, (B) tannic acid tablets, each tablet 1 g, dissolve 1-2 tablets in 100 mL of those gargle solution in embodiments of 3-1.

When gargle solution containing tannic acid is selected and used, spit it out after gargle, to rinse mouth again by using water or the acid gargle solution in embodiment of 3-1.

### EXAMPLE 3-4

### HPLC Acidic Mobile Phase to Elute Bitter Alkaloids and Glycosides, an Explanation of the Mechanism of Dispelling Bitter

The adhesion of traditional Chinese medicine ingredients onto oral mucosa can be ascribed as their nonspecific affinity, and their association with the taste bud receptors as their specific affinity (for the specific affinity see embodiment 6). The two kinds of affinity are different and interrelated. In vitro, the said non-specific affinity on mucosa can be correlated somehow to the relative retention time on HPLC C18 columns (also see embodiment 4 for non-specific affinity), as both the C18 and cell membrane have some similarities in structure, i.e. the similar fatty acid esters. If the acidic gargle solutions can help the bitter ingredients to be washed off from oral mucosa and taste bud receptors, then the acidic mobile phase should also be able to get a similar elution validation on HPLC C18 columns. Namely, so long as the pH value of the mobile phase decreases (more acidic), more H+ ions will associate with the bitter ingredients, and thus raise their polarity, so as to shorten their retention time (i.e. faster dissociation on C18), a quicker washing off will be observed.

alkaloid RN, glycoside RO+H+→alkaloid RNH++glycoside ROH+

A sharp decrease of the retention time of Berberine (coptis chinensis) and Sophora flavescens alkaloids on HPLC column was observed, when the acidity in the flowing solution increased (lower pH value in mobile phase). That meant the medicine ingredients were sooner washed off. But for the ingredients of glycosides, the effect of speeding up elution is less obvious.

Raised the temperature of HPLC column to 40-50° C., the effect of acid speeding up elution is better observed.

### EXAMPLE 4-1

### The Oil-Water Emulsion Formulation for Post-Medication Gargle

Many ingredients in traditional herbal medicine, e.g. the traditional Chinese medicine, are lipophilic compounds. These characteristics have been used in their extraction process. Some commonly used drug edible accessories are lipophilic as well. Owing to the non-specific affinity between the lipophilic ingredients and the lipophilic accessories, if the accessories are used in gargle preparation, they will attract and draw the bitter ingredients and be able to speed up the bitter ingredients to elute from the mouth. These accessories include: edible oil, essential oil, citric acid esters, glycerin, polyethylene glycol, ethanol, combined with surfactants of soya lecithin, Tween 80, Poloxamer, and C12 alkyl sulfonic acid sodium salt, and with cyclic dextrin, Chitosan, curdlan, and carboxymethyl cellulose CMC.

The emulsion for gargle use mainly composed of cooking oil 30-50%, certain amount of essential oil, PEG, or glycerine etc, water 70-50%, food/medical surfactants such as 0.1-0.5% lecithin, Tween-80 2-10%, or 0.5% carboxymethyl cellulose CMC, or Poloxamer, a small amount of edible acid 1-2.5%, and a proper amount of oral freshener, sweeteners, flavors, and so on. The cooking oil should be separately packaged, mixed with the aqueous solution before using, and thus formed emulsion could be effective in 1-2 weeks. If individuals do not like this kind of fatty formulation, formulations with other lipophilic agents can be chosen.

This emulsion formulation was personally tested with the bitter medicine in Embodiment 1. It proved that the small oil droplets in emulsion would compete with the taste bud receptors and oral mucosa for the lipophilic ingredients in the residual medicine. Its gargling resulted to a quicker dissociation from the taste buds and less adhesive to the mucosa for the ingredients to move. By using the oil-water emulsion gargle to rinse mouth 2-3 times, the bitter tastes from gentian, Berberine, and Sophora flavescentis was largely cleared respectively, while the bitter taste from Sophora flavescentis still partially stranded due to a small residual amount of bitter ingredients stubbornly stuck on the throat mucosa. To remove this kind of small and stuck bitter taste, the formulations in embodiment 5, 6, and 7, i.e., the absorbent, chew gum, and candy, has to be applied respectively to rinse out, and/or to rinse-swallow. While for the other slightly bitter herbs, such as gardenias, akebia, Viola yedoensis Makino, madder, and scutellaria, by using the oil-water emulsion gargle 1-2 times rinse, the stranded bitter taste could be completely clear, and taste returned fresh.

### EXAMPLE 4-2

### Lipophilic (Organic) Mobile Phase to Elute Bitter Alkaloids and Glycosides on HPLC C18, an Explanation of the Mechanism of Dispelling Bitter

The lipophilic characters of the bitter ingredients have been described in Embodiment 3-4. In fact, not only the acid but also the lipophilic accessories will compete with C18 for the medicine ingredients due to non-specific affinity. The function of organic phase in HPLC C18 column has been generally verified. In our HPLC C18 test, into the aqueous mobile phase a portion of lipophilic accessories such as glycerin and alcohol was added. The retention time of the bitter ingredients such as alkaloids and glycosides from the said herbal medicines were thereby considerably reduced. More ingredients from the C18 column were eluted.

Raised the temperature to 40-50° C., the elution effect was better.

Embodiment of 5-1, Adsorbent formulation for post-medication gargling, in the form of powder, capsules, or rapidly disintegrating tablets

In post-medication some ingredients causing bitter taste in traditional medicinal are locally and generally so toughly adhesive to the mucous membrane in the mouth that the formulations of acid, oil-water emulsion, and enzyme-protein as gargle preparation can still not completely remove its bitterness. Should now be considered an adsorbent formulation, of which activated carbon/activated bamboo charcoal powder, macro-porous resin and other adsorbents are the mostly used agents. As compared to the formulation of acid solution, water/oil emulsion, and enzyme/protein, the adsorbent formulation is stronger and of broader spectrum of dispelling, their applied formulation is partially shown in table 2. Its dosage form, in addition to powder also can be made of garble capsules, gargle tablet with activated carbon-macro porous resin. For example, drug edible activated carbon 0.4 g, activated bamboo carbon powder 0-0.2 g, and/or 0.2 g macro porous resin, hydroxypropyl cellulose 0.1 g, malic acid 0.02-0.06 g, xylitol or mannitol 0.2 g, proper amount of fragrance, surfactants, and effervescent agents such as sodium bicarbonate and citric acid. After mixing, make the capsules, tablets or bagging. Take 1-2 capsules, tablets or 200-400 mg powder for each gargle.

By means of the adsorbent formulation, the mouth gargling demonstrated that by chewing, tongue mixing, xylitol or mannitol dispersing, as well as effervescent rapidly disintegrating, the activated carbon-macro porous resin in capsules, pills, or powder rapidly adsorbed and competed with taste buds for the bitter ingredients. It resulted to a quick and complete dispel of the bitter-foul tastes entirely. Tests also showed that the combination of adsorbent formulation with the embodiment 3 lipophilic acid solution formulation in sequence would be an optional gargle method. The strong bitter-foul taste can be immediately and thoroughly removed by the combined formulation: applying the adsorbent formulation for gargle first and then applying the lipophilic acid formulation secondly.

Embodiment of 5-2, a tooth-paste gel formulation containing adsorbents for post-medication gargle

For ease of use, the activated carbon, macro-porous resin, and other effective adsorbents can be made into a toothpaste gel sealed in a soft special tube. Squeezing the fresh gel out to the mouth and by tongue stirring, the activated carbon and adsorbents would evenly coat on the surface of the mucosa of the tongue and oral cavity. They adsorbed, and competed with the taste bud receptor for, the residual bitter ingredients to dispel bitter taste. As a sample, the gel's components can be: activated carbon 35%, activated bamboo charcoal (or macro-porous resin) 15%, humectant glycerine 16%, thickener sodium carboxy methyl cellulose CMC 1.5%, soya lecithin 0.1-0.5%, or C12 alkyl sulfonic acid sodium 0.8%, sodium saccharin, 0.1%, proper amount of flavor ect. Its adsorption effect is slightly inferior to the above-mentioned embodiment of 5-1.

### EXAMPLE 5-3

### In Vitro Experiment to Prove the Adsorption of Bitter Ingredients by Adsorbents

Adsorbing objects were the decoctions of, without any post processing and additives, Sophora flavescens, Gentian, Berberine, Phellodendron, Coptis, Caulis clematidis armandii, Gardenia, Viola yedoensis makino, Real elm, Madder, Skullcap, and a Berberine (Berberine, about 0.8 mg/mL) hydrochloride solution. Each 0.3 mL decoction was added 2.7 mL water to dilute to 1/10. Adsorbents used: activated carbon, macro-porous resin, and washed bamboo charcoal powder, 200 mg each.

Several controls and blank samples were prepared, controls: just the 1/10 diluted decoctions without any absorbent added.

Blank samples: just water, and water mixed with the adsorbents without any decoction or medicine added.

Adsorption: all samples were mixed on a vortex mixer vortex 30-40 seconds, then 4000 RPM centrifuge 12 minutes or more. HPLC analyze the supernatant.

Results displayed: Viola yedoensis makino, only 95% was adsorbed in the supernatant of activated carbon, and there was 5% residue absorption. Others are completely adsorbed. On their upper clear liquid, the medicine component characteristic peaks, such as alkali and glycosides peaks etc, completely disappeared. This proved that the activated carbon and macro-porous resin were effective for dispelling the bitter ingredients.

The washed bamboo charcoal powder, without activation, could effectively adsorb coptisine and Matrine, whereas could only partially adsorb gentian glycosides and skullcap baicalin.

This experiment demonstrates that activated carbon and macro-porous resin adsorbents can be a strong clearing agent, whereas the bamboo charcoal powder needs further activation.

### EXAMPLE 6-1

### Enzyme-Protein Formulation for Post-Medication Gargling

Powder or Fast disintegration tablet of protein gargle gargle or powder , medication use Lot of the bitter ingredients in medicine, such as glycoside and glucoside class and denoted as RO, are substrates for both taste bud receptor and some specific enzymes. They can associate either with the taste bud receptors on the tongue to produce a bitter taste, or with a certain kind of enzymes due to their specific affinity. Hence a proper enzyme in excess amount added in mouth will compete with the bud receptors for the same bitter ingredients RO:

RO·bud receptors+enzyme→RO·enzyme+free taste buds receptors

These kind drug edible enzymes can be: α-,β-glucosidase, cellulase, amylase, glucose enzymes, hemicellulase, xylanase, rhamnosidase, galactosidase, Lactose enzymes etc.

In addition, a lot of the bitter ingredients from traditional medicines are lipophilic (see also embodiment 4). When they meet some kind of proteins, they may be encapsulated by the hydrophobic cave region in the tertiary structure of a protein. Owing to this kind of non-specific affinity, the lipophilic ingredients in residual medicine will combine with the hydrophobic cave through a hydrophobic bonding. Thus the protein molecule will compete with the taste bud receptors for the bitter ingredients, and draw the ingredients away. Thereby the protein will speed up the dissociation between the ingredients and the taste bud receptors:

RO·taste bud receptors+protein→RO·protein+free taste buds

This kind of available protein includes: whey protein, sodium caseinate, egg albumin, bovine serum albumin, milk powder, isolated soy protein, gelatin, collagen, or hydrolyzed protein products. This also includes some non-protein cellulose derivative such as hydroxypropyl cellulose, as well as β-Cyclodextrin, Chitosan, and so on.

This kind of gargle preparation includes a formulated protein powder, powder in capsules, and/or, a formulated fast disintegrating tablet made of protein. They are mainly formed by these enzymes and proteins. For example, cellulase 0.15 g, hemicellulase, 0.15 g, xylanase enzyme 0.1 g, albumin powder or whey protein 0.5 g, malic acid 0.02-0.06 g, xylitol or mannitol 0.3 G, cellulose derivative 0.2 g, proper amount of effervescent, surfactant, β-Cyclodextrin, Chitosan, as well as a proper amount of oral freshener, flavor, sweeteners. After mixed, encapsulating, tabletting or bagging it, take 1-2 capsules/tablets or 200-400 mg powder for each dose.

Oral tests showed that owing to the fast disintegration, the dispersing agent of mannitol/xylitol and the effervescent, and tongue stirring, a paste of enzyme and protein evenly coated on the surface of the tongue and on the mucosa in oral cavity. The enzyme and protein compete with the taste bud receptors for the bitter ingredients from the residual medicines. It promptly removed the bitter taste. After gargling with the formulated protein powder or tablet, a lipophilic acid solution as described in embodiment of 3 can be applied for further mouthwash. The strong bitter/foul taste in post-medication of traditional medicine will be quickly removed through these two steps gargle.

For the very strong bitter medicine, tests also showed that a sequential combination of adsorbent formulation in embodiment of 5, followed with the protein formulation in embodiment of 6, and then with the lipophilic acid solution formulation in embodiment of 3, would be an optional gargle method. The strong bitter-foul taste can be immediately and thoroughly removed by the combined gargle formulation: applying the adsorbent formulation first, then applying the enzyme-protein formulation second, and then applying the acid formulation which will rinse all out, including rinsing out the remained carbon powder and other gargle components. The oral cavity so returns back to its normal state.

This invention also provided a specially designed dual-bottle container, with a small size to benefit carrying on. One bottle holds water or diluted acid (pH about5-7) for individual to suck up and finally rinse out the residual gargle preparation in mouth. The other bottle is just a container for holding the spat liquid.

### EXAMPLE 6-2

### In Vitro Dialysis Experiment: there is a Possible Bonding Between Bitter Ingredients and Some Proteins, Enzymes

The possible hydrophobic bonding between bitter ingredients RO and protein molecules can be explored by using membrane dialysis:

free RO in decoction+protein→RO·protein

free RO in decoction→membrane→RO detected

Dialysis bag was purchased from Beijing Scientan science and technology company, molecular weight range 8-14 KD. The bag was immersed in 10 mL water in a beaker. The pore size on the membrane inhibited the protein molecules from penetrating.

Into the first group (control samples) bags added in the sophora flavescens, gentian, and scutellaria decoctions and Berberine in aqueous solution respectively, 0.2 mL each, and diluted with water to 2 mL total volume each. To simulate the Chinese medicine, in the 4 medicine decoctions used as sample and control sample, there was no additive and without any post processing.

In the second group of 4×5 bags, four different protein powders were added respectively: whey protein, milk powder, chicken ovalbumin, bovine serum albumin, 5 bags for each protein, 400 mg powder in each bag. As the last step of sample preparation, 2 mL of 1/10 diluted decoction solutions were added into the protein bags, 4 different protein bags for each medicine decoction to add in. Into the remaining set of 4 different protein bags from above, 2 ml water was added instead without any medicine decoction. These protein bags were used as blank or background.

Scheduled sampling from the 10 mL water outside the bag, and followed by HPLC quantitative analysis. Due to permeation membrane diffusion, the medicine ingredients' concentration in the solution outside the bag was correlated to the concentration of the corresponding free ingredients inside the bag.

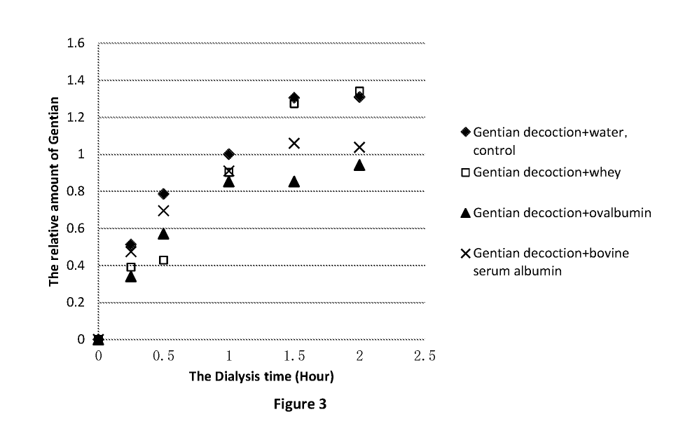

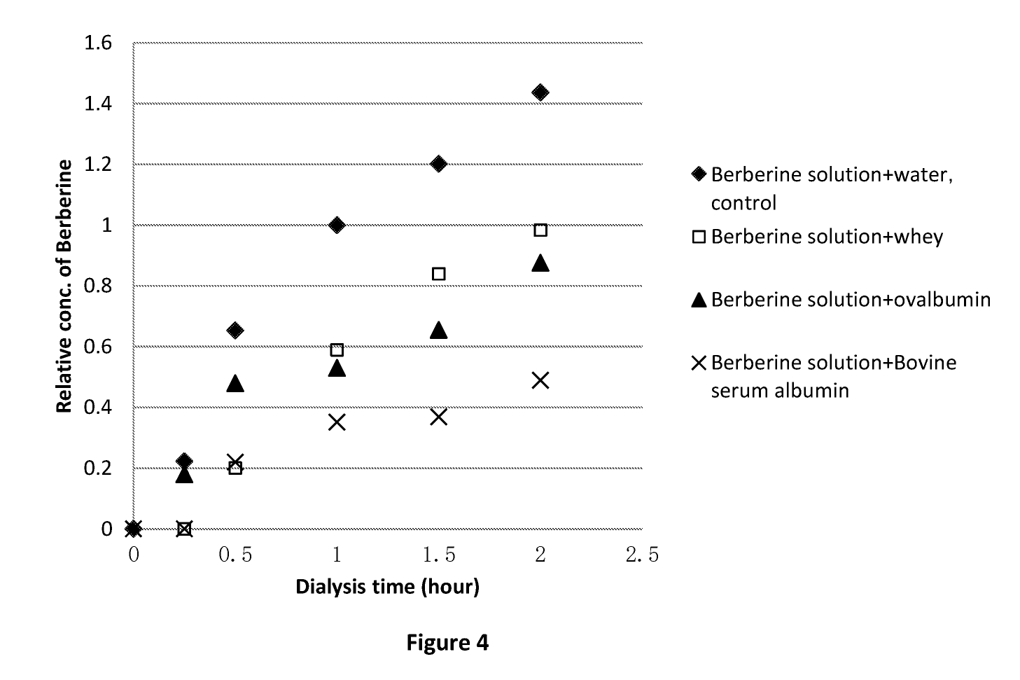

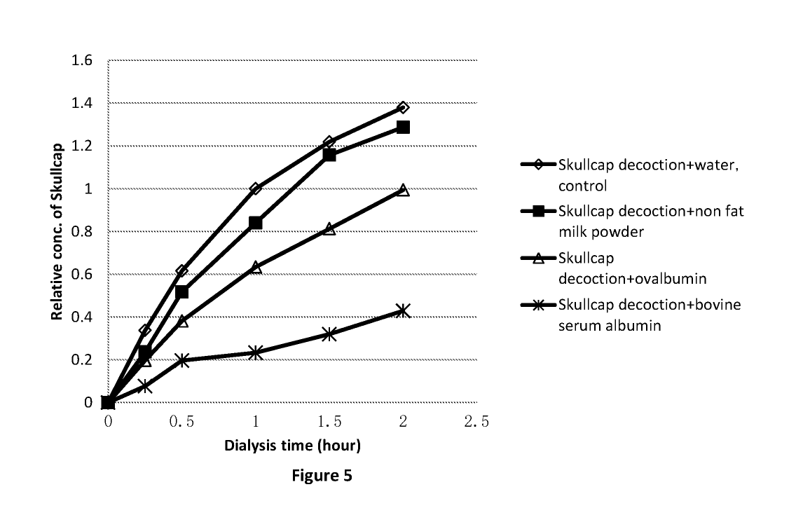

Results were shown in FIG. 2 (Sophora flavescens), FIG. 3 (gentian), FIG. 4 (Berberine), and FIG. 5 (scutellaria baicalensis Georgi). From FIG. 2-5 it can be seen that: when protein was applied inside the bag, the sample concentrations outside permeation membrane were lower than that outside the control.

The dialysis results demonstrated that inside the bag the protein hydrophobic cave encapsulated the free ingredients in the herbal decoction through a possible hydrophorbic bonding. Hence less free ingredients of medicine can go, through a permeation membrane diffusion, to outside bag.

In mouth, the protein molecules will compete with taste bud receptors and with the oral mucosa for the bitter ingredients, which will be encapsulated and drawn away by protein molecules. Among them, the encapsulation or hydrophobic bonding ability of bovine serum albumin seems strongest. Thereby enzyme-protein formulation can clear the bitter medicine.

The specific affinity or bonding, between the related enzyme and their substrates, i.e. the ingredients including glycosides and glucosides RO, should also be seen by means of dialysis. However, many of the dialysis membrane are made from cellulose re-production, which are vulnerable to the attack from enzymes such as α-, β-glucosidase, cellulase and hemicellulase. Seems to be only an indirect experimental description of the substrate by using an enzyme-catalyzed hydrolysis of glycoside and glucoside.

Cellulase 7.5 mg and hemicellulase 7.5 mg mixture, a total of 4 mixtures of this kind were made. Two of the 4 were added medicine gentian decoction, and the other two were added scutellaria decoction, 1 mL each. To simulate the Chinese medicine, both decoctions were supplied without any post processing. One gentian and one scutellaria were set at 37° C. for 2 hours, and other two were set at 45° C. overnight respectively.

Control samples were made directly from the medicine decoction, without adding any enzymes. Blank were made just adding 1 mL of water into the same enzymes mixture.

HPLC analyzed both the residual glycosides and the hydrolized aglucone. All the samples showed significant enzymatic hydrolysis, the aglucone concentrations increased sharply. This illustrated the enzyme-glycoside (the substrate) association did exist. In mouth the enzyme compete with the taste bud for bitter glycoside and glucoside in traditional Chinese medicine, thus drawing and eluting them away to dispel the bitter taste.

### EXAMPLE 7

### The Formulation of Acidic Gum and Hard/Soft Candy

By using polyvinyl acetate as raw materials for gum, into each piece of gum base added 100-200 mg of malic acid and its salt,100-200 mg xylitol or mannitol; into each piece of the hard/soft candy base, added 100-200 mg malic acid or citric acid and the salts, or into each toffee base added lactic acid and its salt 100-200 mg, also added proper amount of surfactants, adsorbents, lipophilic excipients, oral freshener, mixing and pressing, an acidic gum, candy or toffee was made with pH 3-9 in solution. For some medicine with weak bitter or mild bitter taste, instead of using a gargle formulation, 1-2 pieces candy chewing or taken in post-medication will help dispelling the mild bitter and peculiar-foul taste.

In some other instance that a strong bitter medicine like radix sophorae flavescentis decoction was taken and even both the pre-medication gargle and the post-medication gargle were applied, there might be still some bitter residue stuck stubbornly on the mucosa in the throat part, causing bitter taste. In such kind of case, usually a gargle preparation will be hard to reach and dispel it on the throat mucosa. A mild acidic candy or protein solution can be used in addition to the gargling. Should chew the mild acidic gum or candy, and swallow in a small amount of mild acidic liquid thereof, or swallow in a small amounts of protein gargle solution, the bitter ingredients in the throat mucosa will be cleaned and rinsed down to stomach.

### EXAMPLE 8

### Initial Human Tests

Selected several commonly used bitter taste decoction of traditional Chinese medicine as a test target, respectively, they include Sophora flavescens, gentian, cork, Berberine, scutellaria, Kawaki pass, Berberine (Berberine), bitter gourd, Burnet, gardenia, madder, and so on. Among them, Sophora flavescens was used as a major test object of dispelling bitter taste. Sophora flavescens has high bitter strength, and is widely distributed on the taste buds and oral mucosa, binding firmly resulted to extended bitter taste. It is difficult to remove, called as “extremely bitter”. If this invented formulation of dispelling bitter could not remove the bitterness of Sophora flavescens, then the invention would be not perfect and uncompleted.

Pre-medication gargle preparation experiment

Before taking medicine, the gargle preparation followed embodiment 2.

Post-medication gargle preparation experiment

After taking medicine, the gargle preparations followed the embodiment 3 through to 7.

The general gargle method used

More than 10 people volunteered for the experiment, mostly adults, there are also young people. Under the guidance of a Chinese medicine physician to make a mouthwash dispelling bitter taste, the attempts are as follows:

A. Bitter medicine taste: practiced the same as that in embodiment 1. When time began, had 2-5 mL of bitter medicine decoction in mouth and mixed it by the tongue, recorded the bitter feeling, spat it out after about a minute, then water gargling and spat it out. Started right again the second time to observe the bitter retention and its relationship with time, during each 5-minute took a water gargling again.

B. pre-medication gargling effect: generally dozens of mg or 0.1-0.4 grams of polysaccharide gum powder was spread on the tongue, chewing in the mouth, mixed and stirred it by tongue, spat it out after a few minutes. Then immediately had 2-5 mL of bitter medicine decoction in mouth, and followed the same procedures as stated in A above or as that in embodiment of 1. Compared the bitter feeling result from B above to that result from A, and determined the pre-medication gargling effects in terms of masking the taste buds before they took effect.

C post-medication gargling effects: had 2-5 mL of bitter medicine decoction in month, and mixed, stirred with tongue about 1 minute, spat it out, left a bitter taste in the mouth. Then immediately gargled with acidic solution formulation, rinsed 1-2 times, started the time clock to record the retained bitter feeling. If limited effects of dispelling bitter occurred, then followed by further testing of oil-water emulsion formulation, then tested enzyme-protein formulation, and adsorbent formulation powder mouthwash. At end, compared the result to A or that in embodiment of 1, to determine the dispelling bitter effects of post-medication gargling.

Preliminary test results of human trials are:

The bitter dispelling effects of pre-medication gargling:

(1) For medicines of general bitter taste, formulated oil-water emulsion, formulated protein powder agents, formulated chitosan adhesive agents for bio-mucosa, formulated xanthan gum and other viscous gums, their powders or solutions, all can be used for pre-medication gargling. They can partially cover the taste buds in pre-medication and protect them during medication. To a quite degree, they reduce the bitter sense strength caused in medication process, and obviously shorten the stranded time of bitter taste.

(2) For medicines of strong bitter taste, such as Radix sophorae flavescentis, Gentian, Berberine, Phellodendron etc, they require a pre-medication gargle formulation of polysaccharide gum with higher viscosity in the form of powder and thus more amount as needed can be directly laid on the surface of tongue, such as table 1 listed formulations in xanthan gum, guar gum etc.

(3) For a relatively flat portion of the oral mucosa, for example, top of tongue, back portion of tongue, part of throat, the palate mucosa, and sublingual mucosa etc., where the taste buds and mucous membranes can more easily maintain the viscous gum/gel thin layer coating, thereby the bitter intensity and stranded bitter feeling remain much less there.

While on the left and right sides of the tongue, as well as on parts of the throat mucosa, because of peristalsis, friction, and the erosion by drugs' flow, the viscous gum/gel thin layer coatings loosen and lost there. On these portions, some of the bitter taste buds are still likely to be exposed to the bitter ingredients' direct attack. Hence, there partially failed to dispel the bitter taste, the stranded bitter feeling will be more re-distributed and remained there.

The bitter dispelling effects of post-medication gargling:

(1) Generally, medicines of mild bitter taste, such as: Caulis clematidis armandii, Burnet, Scutellaria, and Charantia, a gargling and rinsing with lipophilic acid formulation or oil-water emulsion formulation of 5-20 mL will be enough to remove entire bitter taste. Mouth will immediately restore normalcy.

Instead of an acid formulation, a vinegar rinse can also play a similar role, but vinegar has its limitations: the vinegar itself is also a traditional medicine, which contains volatile substances such as aldehydes, ketones, and esters. It may have potential effects on the prescription medicines, and its effects of dispelling bitter are less powerful than the formulated acid solution. In addition, a daily repeated gargle with vinegar is far less than using a variety of different lipophilic acid formulation of different oral freshener and different fruit-flavored mouthwash.

Prior to the vinegar formulation to be adjusted according to this invention, e.g. its pH, its concentration, the lipophilic excipients and oral freshener applied, common vinegar is not recommended as a gargle solution to remove bitter taste.

(2) For medicines of strong bitter taste such as Gentian, Berberine, Phellodendron, Coptis etc., or for those who have more sensitive taste buds than others, three steps gargling may be needed. In the first step, gargle and rinse with lipophilic acid formulation. Then in the second step, gargle and rinse with formulated enzyme-protein gargle powder, powder in capsule (or fast disintegrated tablets), or with oil-water emulsion formulation. Afterwards, again gargle and rinse with lipophilic acid solution formulation. All these can completely dispel the stranded bitter/foul taste, and oral cavity returns to normal.

(3) Medicines of strong bitter taste, such as radix sophorae flavescentis, gentian etc., after dosing they stubbornly stuck in the mouth for a long time. The application of lipophilic acid solution formulation, enzyme-protein formulation, and oil-water emulsion formulation, or a combination thereof, works well and properly, but always are not so sufficient to remove all the bitter taste, particularly for Sophora flavescens. In these cases, an adsorbent formulation, such as powder of activated carbon (and a little of activated bamboo charcoal of ultrafine particles) and/or powder of macro-porous resin, and other adsorbents, mixed with baking soda effervescent, can completely eliminate the bitter taste, then mouth returns to normal. Baking soda and proper acid here plays a role of effervescent agent.

In addition, sometime the bitterness left on the deep throat mucosa, it will need to chew acid+candy or acid+gum mixture and to swallow a small amount of the solution or protein solution to rinse and clear the throat, to remove the last bitter roots.

Gargle preparation and method of the invention not only remove the bitter, but also remove some of the odor and protection of taste

Based on multiple human trials for dispelling medicines' side effects on taste sense, it was found that not only the bitter taste, but also the peculiar-foul taste were removed as well. Furthermore, the temporarily dulled taste sense as a side effects caused by multiple dosing of the traditional medicine was also improved a lot.

(1) Some people joined the trials who are currently taking Chinese herbs decoction for healthy purpose, immediately after swallowing the drug decoction, an acid mouthwash was performed under guidance. It was then reported that the gargle not only removed the bitter taste of the traditional Chinese medicine, but also the unique peculiar-foul taste induced by traditional Chinese medicine was also removed.

(2) Some people joined the trials who are currently taking Chinese herbs decoction for healthy purpose. The medicine taken was of neither bitter, nor peculiar-foul taste. Whereas as its temporary side effect, the medicine thickened the tongue coating, dulled taste buds, and caused appetite loss. This kind of dulled taste was a side effect of complains historically. However to use the invented gargle instead, the thickening of tongue coating, dulled taste buds, and appetite loss were improved a lot, and the taste sense returned closer to normal.

(3) Since the gargle preparation for post-medication has multiple formulations and their combinations thereof, its function allows its usage can be extended to some area other than medicine, such as post-food eating gargling, post-drinking gargling, even a gargle for bad breath smell. The practice showed that it was successful for people to use this invented post-medication gargle method to dispel odd tastes, such as post-dinner chili spicy taste, pungent taste from garlic, onions or other spices, fishy taste, protein foul taste, and even bad breath smell. These taste and/or smell was produced from the residual materials stuck on the oral mucosa, which can be removed quickly and effectively by using the invented post-medication gargle preparation.

The above findings and extended applications are consistent with the mechanism of the present invention. All the strange taste, bitter taste, spicy and pungent taste, peculiar-foul taste, and the taste attenuation etc. can be caused by either medicine or variety of food. The invented gargle preparation and methods can protect widely various taste buds in the mouth by thin layer mask and/or rinse out, which will otherwise be directly and sustained attacked by some ingredients in medicine, or food.

So, for all those which is taken orally and makes oral taste in non-normal state, for example various traditional medicine, alternative medicine, traditional Chinese medicine, Western medicine, nutrition, or food etc., this invention can be used to remove the non-normal tastes such as bitter, peculiar-foul, spicy taste, pungent taste, some smelling, or their other side effects on taste buds like taste attenuation, to keep taste normal and oral health.

All documents mentioned in the invention are referenced in this application as a reference, as each article be taken alone as a reference. Should also understand, after reading the above teaching of the invention, the field technician can make changes or modifications to the invention. These equivalent forms of the same fall in the scope of claims attached to the application as defined by.

What is claimed is:

1. A gargle preparation for removing the bitter taste in oral medication or food comprising at least one of two preparations as follows:
the first preparation, which is suitable for gargling right before taking oral medication or food forming a protective thin layer of shielding on the mucosal surface in the oral cavity to reduce the degree and time of the contact between the mucosal surface and the bitter ingredients as well as other taste side-effect factors in the medicine or food; and
the second preparation, which is suitable for gargling right after taking oral medication or food, facilitating the rapid dissociation and elution of the bitter ingredients and other taste side-effect factors from the mucosal surface in the oral cavity.

2. The first preparation of a gargle preparation as described in claim 1 comprising at least one ingredient from the group of pharmaceutical protein, lipophilic agent, mucosal adhesive, polysaccharide gum, oil-water emulsion, cellulose derivative, adsorbent, pharmaceutical hydrogel, and oily gel; and
The second preparation of a gargle preparation as described in claim 1 comprising at least one ingredient from the group of pharmaceutical acid, lipophilic agent, pharmaceutical protein or enzyme, mucosal adhesive, cellulose derivative, oil-water emulsion, adsorbent, surfactant, effervescent agent, polyvinyl pyrrolidone, and pharmaceutical gel base.

3. A gargle preparation as described in claim 2 has at least one of five characteristics as follows:
1) the weight percentage of the polysaccharide gum or the pharmaceutical hydrogel in the first preparation is from 0% to 100%;
2) the weight percentage of the pharmaceutical acid in the second preparation is from 0% to 100%;
3) the weight percentage of the adsorbent in the second preparation is from 0% to 100%;
4) the weight percentage of the pharmaceutical protein and enzyme or the weight percentage of the polyvinyl pyrrolidone in the second preparation is from 0% to 100%; and
5) the weight percentage of the lipophilic agent in either the first preparation or the second preparation is from 0% to 100%.

4. A gargle preparation as described in claim 2 comprising one or more ingredients from the group of pharmaceutical acid-base regulator, surfactant, effervescent agent, dispersing agent, suspension agent, emulsion agent, flavoring agent, pharmaceutical gel base, essential oil, preservative, flavor, and oral freshener.

5. A gargle preparation as described in claim 1 being formed in a dosage form of powder, capsule, rapidly disintegrating tablet, solution, suspension, emulsion, syrup, liquid spray, paste, cream, ointment, oral hydrogel, chewing gum, chewing candy, or oral adhesive patch.

6. A gargle preparations as described in claim 1, wherein the first preparation is taken and spat out immediately or within a few minutes before having oral medication or food and the second preparation is used immediately or within a few minutes after having oral medication or food.

7. A gargle preparation as described in claim 1 having means for reducing oral taste abnormalities caused by oral medication or food.

8. A gargling method of using the gargle preparation as described in claim 1, comprising at least one of two steps as follows:
1) right before taking oral medication or food, gargle the user's oral cavity for a few minutes by using the first preparation of the gargle preparation; and
2) immediately after taking oral medication or food, gargle the user's oral cavity several times by using the second preparation of the gargle preparation.

9. A package of the gargle preparation as described in claim 1 comprising the first pack that contains the first preparation and the second pack that contains the second preparation.
